(19) United States
(12) Patent Application Publication
(10) Pub. No.: US 2026/0339437 A1
(43) Pub. Date: Oct. 29, 2026

(54) USE OF SECONDARY DATA DURING NEURAL DECODING IN BRAIN COMPUTER INTERFACE SYSTEMS

(71) Applicant: Synchron Australia Pty Limited, Fitzroy (AU)

(72) Inventors: Thomas James Oxley, Dover, DE (US); Peter Eli Yoo, Dover, DE (US); Nicholas F. Hardy, Dover, DE (US); Sadegh Ebrahimi, Dover, DE (US)

(21) Appl. No.: 19/657,553

(22) Filed: Apr. 24, 2026

Related U.S. Application Data
(60) Provisional application No. 63/794,893, filed on Apr. 25, 2025.

(51) Int. Cl. G06F 3/01

(52) U.S. Cl. CPC G06F 3/015

(57) ABSTRACT

Decoders for use in brain-computer interfaces (BCI) to decode a neural signal and use secondary data from an individual using the BCI into some actionable command that allows the BCI to interact with a device.

### CROSS-REFERENCE TO RELATED APPLICATIONS

This application claims priority to U.S. Provisional Patent Application No. 63/794,893, filed on Apr. 25, 2025, and titled “USE OF SECONDARY DATA DURING NEURAL DECODING IN BRAIN COMPUTER INTERFACE SYSTEMS,” which is hereby incorporated in its entirety.

### BACKGROUND OF THE INVENTION

Decoders for use in brain-computer interfaces (BCI) detect a recorded or detected neural signal from an individual using the BCI and decode the neural signal into some actionable command that allows the BCI to interact with a device coupled to the BCI. Current BCIs primarily focus on a region of the brain, e.g., a motor cortex, to determine neural activity for motor intention and decode the associated electronic signal to produce an output signal that ultimately controls applications such as cursor movement, typing, and robotic actuation. While traditional EEG-based error detection (e.g., error-related negativity, ERN) has been studied, it suffers from low spatial resolution, high noise, and reliance on explicit behavioral responses. Existing BCIs lack a robust mechanism to detect and use validation context data comprising information associated with the individual BCI user to assist in improved application of the output signal. There remains a need for using supplemental information to augment interaction between a BCI system and its user.

### SUMMARY OF THE INVENTION

The present disclosure includes method of decoding an electronic signal that is generated by a neural interface device configured to detect a brain activity of an individual and using secondary data to determine how to decode the signal such that decoding of the signal is affected by factors related to the individual.

In one example, the disclosure includes methods and systems of assisting an individual using a brain-computer interface to engage an electronic device. For example, the method can include transmitting an electronic signal from a neural interface device of the brain-computer interface to a processing component of the brain-computer interface; where the electronic signal is associated with neural activity of the individual from a first region in a brain of the individual and where the processing component is configured with a plurality of algorithms including a decoding algorithm and a supplemental algorithm; obtaining a secondary data comprising information associated with the individual and transmitting the secondary data to the processing component; decoding the electronic signal using the decoding algorithm to produce an output signal; and processing the secondary data using the supplemental algorithm to determine whether altering an effect of the output signal on the electronic device is required.

In some cases, the processing of secondary data can require altering an effect of the output signal by causing the electronic device to ignore the output signal or by providing additional information such as to provide additional context to the output signal (such as to convey emphasis, emotion, etc.)

In an additional variation, a method or system can include transmitting an electronic signal from a neural interface device of the brain-computer interface to a processing component of the brain-computer interface; where the electronic signal is associated with neural activity of the individual from a first region in a brain of the individual and where the processing component is configured with a plurality of algorithms including a decoding algorithm and a supplemental algorithm; obtaining a secondary data comprising information associated with the individual and transmitting the secondary data to the processing component; and decoding the electronic signal using the decoding algorithm to produce an output signal; applying the supplemental algorithm to the secondary data to determine whether to alter the output signal prior to transmitting the output signal to an electronic device coupled to the brain-computer interface.

Variations of the methods and systems can include altering the effect of the output signal by terminating transmission of the output signal to the electronic device or by instructing the electronic device to ignore the output signal.

The first region of the brain can comprise a motor cortex region of the brain and, where the secondary data is associated with neural activity of the individual from a second region in the brain of the individual. For example, the second region can comprise a region selected from a group consisting of a supplementary motor cortex, a frontal cortex, a dorsal anterior cortex, and a pre-frontal cortex. The secondary data can comprise a cognitive neural activity. The secondary data can comprise muscle movement data associated with the individual.

In one variation, the supplemental algorithm includes a historical data recorded from the individual during use of the brain-computer interface. The secondary data can be contemporaneous with electronic signal or can coincide in time with the neural activity. The secondary data can be detected using the neural interface device.

In some aspects, the techniques described herein relate to a method of assisting an individual using a brain-computer interface to engage an electronic device, the method including: transmitting an electronic signal from a neural interface device of the brain-computer interface to a processing component of the brain-computer interface, wherein the electronic signal is associated with neural activity of the individual from a first region in a brain of the individual and wherein the processing component is configured with a plurality of algorithms including a decoding algorithm and a supplemental algorithm; obtaining a secondary data including information associated with the individual and transmitting the secondary data to the processing component; decoding the electronic signal using the decoding algorithm to produce an output signal; and processing the secondary data using the supplemental algorithm to determine whether altering an effect of the output signal on the electronic device is required.

In some aspects, the techniques described herein relate to a method, wherein altering the effect of the output signal includes terminating transmission of the output signal to the electronic device.

In some aspects, the techniques described herein relate to a method, wherein altering the effect of the output signal includes instructing the electronic device to ignore the output signal.

In some aspects, the techniques described herein relate to a method, wherein the first region of the brain includes a motor cortex region of the brain.

In some aspects, the techniques described herein relate to a method, wherein the secondary data is associated with neural activity of the individual from a second region in the brain of the individual.

In some aspects, the techniques described herein relate to a method, wherein the second region includes a region selected from a group consisting of a supplementary motor cortex, a frontal cortex, a dorsal anterior cortex, and a pre-frontal cortex.

In some aspects, the techniques described herein relate to a method, wherein the secondary data includes a cognitive neural activity.

In some aspects, the techniques described herein relate to a method, wherein the secondary data includes muscle movement data associated with the individual.

In some aspects, the techniques described herein relate to a method, wherein the supplemental algorithm includes a historical data recorded from the individual during use of the brain-computer interface.

In some aspects, the techniques described herein relate to a method, wherein the secondary data is contemporaneous with electronic signal.

In some aspects, the techniques described herein relate to a method, wherein obtaining the secondary data includes obtaining the secondary data that coincides in time with the neural activity.

In some aspects, the techniques described herein relate to a method, wherein the secondary data is detected using the neural interface device.

In some aspects, the techniques described herein relate to a method of assisting an individual using a brain-computer interface to engage an electronic device, the method including: transmitting an electronic signal from a neural interface device of the brain-computer interface to a processing component of the brain-computer interface, wherein the electronic signal is associated with neural activity of the individual from a first region in a brain of the individual and wherein the processing component is configured with a plurality of algorithms including a decoding algorithm and a supplemental algorithm; obtaining a secondary data including information associated with the individual and transmitting the secondary data to the processing component; decoding the electronic signal using the decoding algorithm to produce an output signal; and applying the supplemental algorithm to the secondary data to determine whether to alter the output signal prior to transmitting the output signal to an electronic device coupled to the brain-computer interface.

In some aspects, the techniques described herein relate to a method, wherein altering the effect of the output signal includes terminating transmission of the output signal to the electronic device.

In some aspects, the techniques described herein relate to a method, wherein altering the effect of the output signal includes instructing the electronic device to ignore the output signal.

In some aspects, the techniques described herein relate to a method, wherein the first region of the brain includes a motor cortex region of the brain.

In some aspects, the techniques described herein relate to a method, wherein the secondary data is associated with neural activity of the individual from a second region in the brain of the individual.

In some aspects, the techniques described herein relate to a method, wherein the second region includes a region selected from a group consisting of a supplementary motor cortex, a frontal cortex, a dorsal anterior cortex, and a pre-frontal cortex.

In some aspects, the techniques described herein relate to a method, wherein the secondary data includes a cognitive neural activity.

In some aspects, the techniques described herein relate to a method, wherein the secondary data includes muscle movement data associated with the individual.

In some aspects, the techniques described herein relate to a method, wherein the supplemental algorithm includes a historical data recorded from the individual during use of the brain-computer interface.

In some aspects, the techniques described herein relate to a method, wherein the secondary data is contemporaneous with electronic signal.

In some aspects, the techniques described herein relate to a method, wherein obtaining the secondary data includes obtaining the secondary data that coincides in time with the neural activity.

In some aspects, the techniques described herein relate to a method, wherein the secondary data is detected using the neural interface device.

In some aspects, the techniques described herein relate to a computer-implemented method for secondary evaluation of candidate action data, the computer-implemented method including: receiving candidate action data indicating an intended action of a user derived from decoded neural signal data of the user; receiving validation context data including at least one of: environmental data associated with an environment in which the user is located, location data associated with a location or movement of the user, behavioral data associated with prior behaviors of the user, or physiological data representing a physical condition of the user; evaluating the candidate action data based on the validation context data to determine whether to alter the candidate action data; generating modified action data based on the candidate action data and the evaluation; and transmitting the modified action data to a computing device to cause the computing device to execute an action.

In some aspects, the techniques described herein relate to a computer-implemented method, wherein the validation context data is generated within a time window associated with a time at which the candidate action data is received.

In some aspects, the techniques described herein relate to a computer-implemented method, wherein the validation context data is generated within a time window associated with a time at which the candidate action data is generated.

In some aspects, the techniques described herein relate to a computer-implemented method, wherein generating modified action data includes altering a type of action of the candidate action data.

In some aspects, the techniques described herein relate to a computer-implemented method, wherein generating modified action data includes modifying a length of time during which a candidate action of the candidate action data is to be performed by the computing device.

In some aspects, the techniques described herein relate to a computer-implemented method, further including transmitting the candidate action data to the computing device.

In some aspects, the techniques described herein relate to a computer-implemented method, wherein the validation context data is received before transmitting the candidate action data to the computing device.

In some aspects, the techniques described herein relate to a computer-implemented method, wherein the validation context data is received after transmitting the candidate action data to the computing device.

In some aspects, the techniques described herein relate to a computer-implemented method, wherein the modified action data is a suppression signal, and wherein transmitting the modified action data to the computing device causes the computing device to stop executing the action.

In some aspects, the techniques described herein relate to a computer-implemented method, wherein the candidate action data includes a confidence value, and wherein the computer-implemented method further includes: determining the confidence value satisfies a threshold confidence value, and wherein transmitting the candidate action data to the computing device is based on the confidence value satisfying the threshold confidence value.

In some aspects, the techniques described herein relate to a computer-implemented method, wherein evaluating the candidate action data based on the validation context data to determine whether to alter the candidate action data includes: applying the candidate action data and the validation context data as input to a machine learning model to cause the machine learning model to generate a determination as to whether a candidate action of the candidate action data is accurate.

In some aspects, the techniques described herein relate to a computer-implemented method, wherein the modified action data is generated in response to the machine learning model determining the candidate action is not accurate.

In some aspects, the techniques described herein relate to a computer-implemented method, wherein the machine learning model further generates an indication of a corrected action.

In some aspects, the techniques described herein relate to a computer-implemented method, wherein generating the modified action data is further based on the indication of the corrected action.

In some aspects, the techniques described herein relate to a computer-implemented method, wherein the modified action data causes the computing device to delay execution of a candidate action associated with the candidate action data.

In some aspects, the techniques described herein relate to a system including: a neural decoder including a computer processor, the neural decoder configured to generate candidate action data from a neural intent signal of a user, wherein the candidate action data represents an action to be executed by a computing device; a secondary data extractor including a computer processor, the secondary data extractor configured to obtain validation context data independent of the neural intent signal; and a secondary action evaluator including a computer processor, the secondary action evaluator configured to generate an alteration to the candidate action data based on the validation context data, the alteration configured to alter an effect of the candidate action data on the computing device.

In some aspects, the techniques described herein relate to a system, wherein the validation context data includes at least one of: environmental data associated with an environment in which the user is located, location data associated with a location or movement of the user, behavioral data associated with prior behaviors of the user, or physiological data representing a physical condition of the user.

In some aspects, the techniques described herein relate to a system, wherein the validation context data is generated within a time window associated with a time at which the candidate action data is received.

In some aspects, the techniques described herein relate to a system, wherein the neural decoder is further configured to transmit the candidate action data to the computing device.

In some aspects, the techniques described herein relate to a system, wherein the secondary action evaluator is further configured to transmit the candidate action data to the computing device.

### BRIEF DESCRIPTION OF THE FIGURES

The drawings shown and described are exemplary embodiments and non-limiting. Like reference numerals indicate identical or functionally equivalent features throughout.

FIG. 1A is a representative illustration of a brain computer interface comprising an implant/electrode device positioned within a brain of an individual.

FIG. 1B shows a remote power supply that can be used to wirelessly charge and/or communicate with an implantable receiver and transmitter unit.

FIG. 1C illustrates a variation of a signal control unit that houses a power supply, processor, and circuity to enable wireless and/or wired electronic communication.

FIG. 1D illustrates an example of a portable signal control unit having a housing with a user interface on the housing.

FIG. 1E illustrates one embodiment of a brain-computer interface (BCI) system.

FIG. 1F illustrates one embodiment of a stent-electrode array implanted within a brain vessel of an individual. The stent-electrode array can be one example of a recording device of the BCI system.

FIG. 1G illustrates a communication conduit connecting the stent-electrode array with a receiver and transmitter unit of the BCI system.

FIG. 1H illustrates a close-up view of an embodiment of the receiver and transmitter unit of the BCI system.

FIG. 1I illustrates an additional variation of an interface system, where the multiple and distinct communication channels of the signal control unit allow an individual BCI user to operatively engage one or more external devices to improve autonomy for the individual.

FIG. 2A illustrates an embodiment of a coiled wire carrying a plurality of electrodes. The coiled wire can be another example of the recording device of the BCI system.

FIG. 2B illustrates an embodiment of an anchored wire carrying a plurality of electrodes. The anchored wire can be another example of the recording device of the BCI system.

FIG. 2C illustrates one embodiment of an electroencephalogram (EEG) device serving as the recording device of the BCI system.

FIG. 2D illustrates one embodiment of an electrocorticography (ECoG) device serving as the recording device of the BCI system.

FIG. 2E illustrates one embodiment of a functional magnetic resonance imaging (fMRI) device serving as the recording device of the BCI system.

FIG. 2F illustrates one embodiment of a functional near-infrared spectroscopy (fNIRS) device serving as the recording device of the BCI system.

FIG. 2G shows a variation in which electrodes of a device are positioned within a vessel of a brain to allow recording from multiple regions of the brain and in deep brain tissue.

FIG. 3A illustrates certain software layers or modules running on a computing device of the BCI system.

FIG. 3B illustrates a basic example of using context to determine which decoder is used to decode a signal in a BCI.

FIG. 4A illustrates one embodiment of a neurofeedback graphical user interface (GUI).

FIG. 4B illustrates another embodiment of the neurofeedback GUI.

FIG. 4C illustrates yet another embodiment of the neurofeedback GUI.

FIG. 4D illustrates an additional embodiment of the neurofeedback GUI.

FIG. 4E illustrates a further embodiment of the neurofeedback GUI.

FIG. 5A illustrates one embodiment of a graphic element displayed to the individual representing a current brain activity of the individual.

FIG. 5B illustrates another embodiment of a graphic element displayed to the individual representing a current brain activity of the individual.

FIG. 5C illustrates yet another embodiment of a graphic element displayed to the individual representing a current brain activity of the individual.

FIG. 6 illustrates an embodiment of a neurofeedback GUI comprising graphic elements displayed in temporal succession.

FIG. 7 illustrates examples of different types of sounds that can be generated by an auditory feedback component.

FIG. 8 illustrates examples of different types of tactile feedback components that can generate tactile feedback that can be felt by the individual.

FIG. 9 is a block diagram of an illustrative environment for providing secondary evaluation of a candidate action according to some embodiments.

FIG. 10 is a block diagram of an environment for acquiring validation context data for secondary evaluation of a candidate action according to some embodiments.

FIG. 11 is an illustrative chart showing a latency window in which a conflict signal may be associated with a motor intent signal according to some embodiments.

FIG. 12 is a block diagram of illustrative data flows for providing secondary evaluation of a candidate action according to some embodiments.

FIG. 13 is a visual illustration of a difference in a false positive rates when using a single-layer BCI as compared to a two-layer BCI according to some embodiments.

FIG. 14 is a block diagram of illustrative data flows for controlling a robotic actuator according to some embodiments.

FIG. 15 is a flow diagram of an illustrative routine for updating a neural intent based on secondary data according to some embodiments.

FIG. 16 is a block diagram of an illustrative computing device for decoding neural signals according to some embodiments.

### DETAILED DESCRIPTION

The present disclosure relates to secondary evaluation of action data generated by a brain-computer interface (BCI) to control a computing device.

Some existing systems providing BCIs generate action data to control a computing device based on the neural signals detected by a recording device. However, these existing systems can be limited in their ability to verify whether the action data aligns with the user's intention, or is generated in error. For example, the recording or interpretation of neural signal data used to generate the action data may be affected by environmental factors or the user's physical condition. Existing approaches for validation include the use of multiple separate decoding systems to interpret neural signal data, however such systems may still fail to account for environmental or physical conditions and continue to generate action data in error.

Such existing systems may, therefore, face significant limitations in the reliability of generated action data, particularly when a condition of the user of the BCI system is changing. For example, neural signal data recorded and used to train a decoder of a BCI for a user may be recorded when the user is in a standardized (e.g., a laboratory or healthcare environment) or known (e.g., the user's home) environment. However, when a user is in an excited state, in motion, in an unfamiliar environment, or otherwise subject to a change in physical or psychological conditions, the neural signal data generated by the user may vary from previously-recorded neural signal data used to train a decoder of the BCI. This difference between the environment the training data is generated in, and the actual conditions users of a BCI experience, can lead to the above-noted lack of reliability in decoded outputs reducing the accuracy of the BCI system output or limiting the usability of the BCI system for the user due to the limited conditions in which the BCI system can generate accurate output.

Some aspects of the BCI system of the present disclosure address some or all of the issues noted above, among others, by providing for secondary evaluation, and validation, of action data generated by a BCI system. The secondary evaluation may be performed by a same or different computing device executing other components of the BCI system (e.g., a decoder). Some or all of the functionality of the BCI system may be implemented in a cloud-based environment, for example using computing resources available via a network connection between a recording device capturing a user's neural signal data and a user device being controlled by the user.

The secondary evaluation performed by the BCI system may be based on various types of validation context data available to the BCI system. For example, the validation context data may include physiological data about the user, environmental data, location or motion data, or any other type of data that may be provided to the BCI system related to the user's current condition when attempting to perform an action using the BCI system to control a computing device. The secondary evaluation may be performed by one or more trained machine learning models that are trained to determine whether action data generated by the BCI system is correct or an error. A machine learning model performing the secondary evaluation may be trained on labelled data indicating whether previously-generated action data correctly represented an intent or was generated in error. In some embodiments, the secondary evaluation may be performed according to a set of deterministic rules defined to adjust BCI-determined action data using additional data. The secondary evaluation system may be executed when action data is generated by the BCI, allowing the computing resources used by the secondary evaluation system to be released when no action data is currently available to be evaluated.

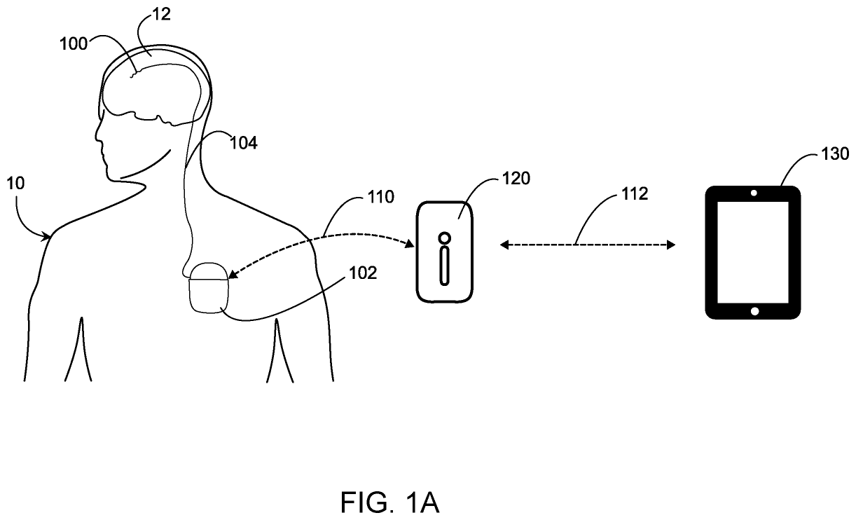

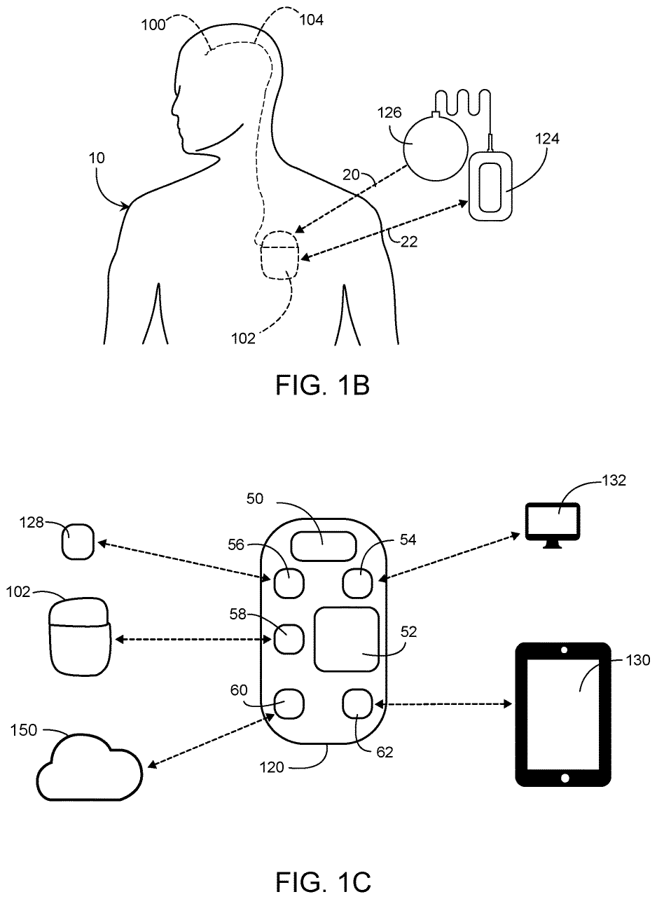

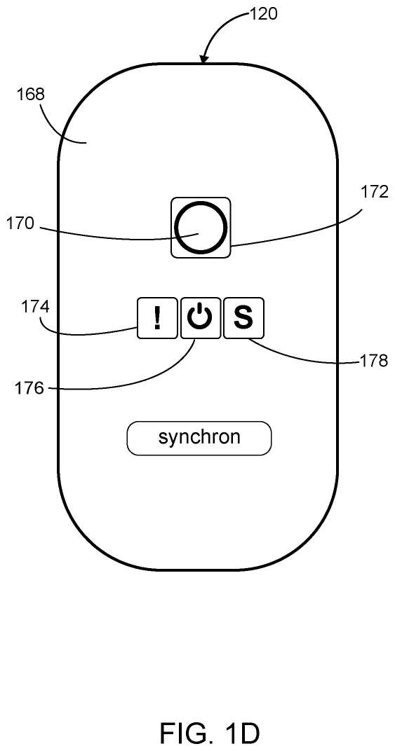

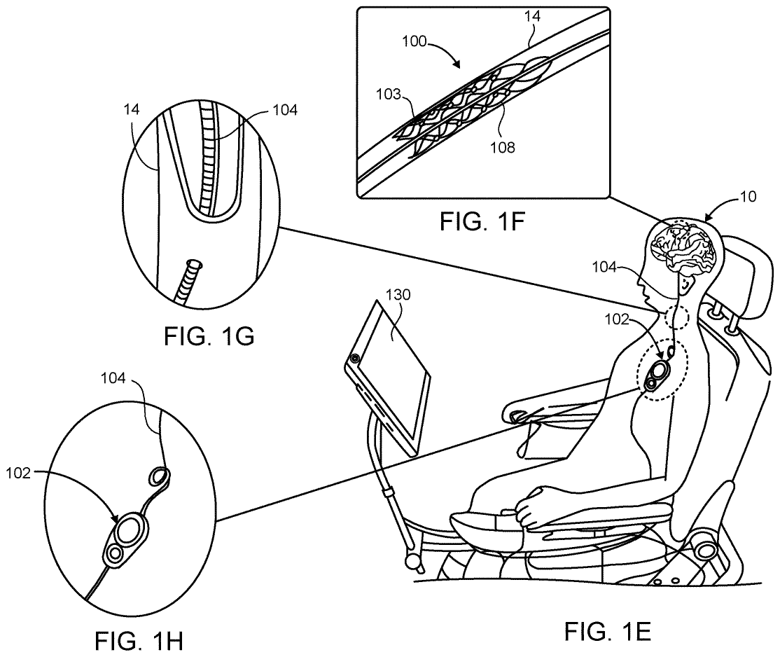

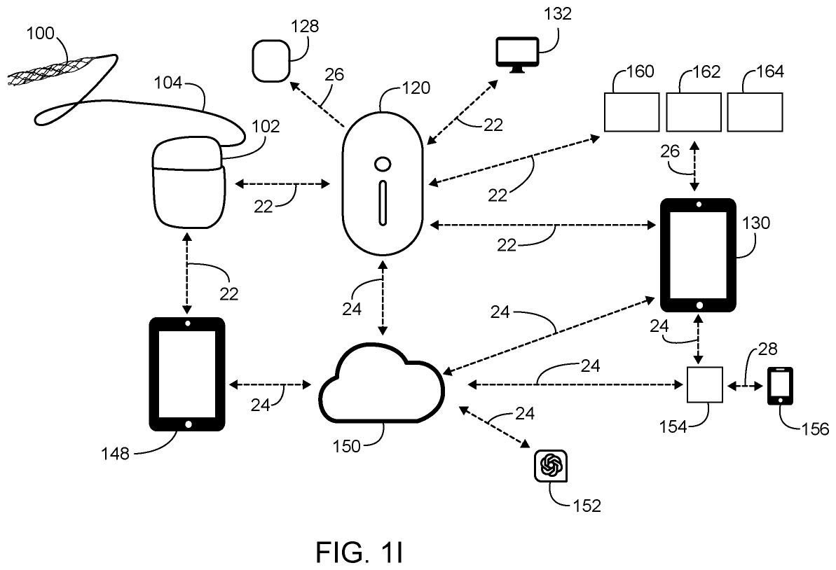

FIG. 1A is a representative illustration of a brain-computer interface comprising an implant/electrode device 100 positioned within a brain 12 of an individual. The implant 100 can be coupled to a receiver and transmitter unit 102, where the receiver and transmitter unit produces an electronic neural signal corresponding to brain signals from the brain of the individual detected by the implant 100. Typically, the implant 100 is coupled to the receiver and transmitter unit 102 via one or more leads 104. However, this communication can occur wirelessly. Moreover, the systems described herein can be used with various other implantable and non-implantable external devices configured to detect brain activity. In addition, the receiver and transmitter unit 102 can comprise an implantable housing where charging is performed through a capacitive or similar configuration from an external charging unit. Alternate variations include a receiver and transmitter unit 102 that is external to the individual 10.

FIG. 1A also shows the receiver and transmitter unit 102 having an ability to communicate with a signal control unit 120 that receives transmissions from the receiver and transmitter unit 102 and is configured to perform signal processing on the electronic neural signal to perform any number of functions for interaction with a host device 130 or a number of host devices. A host device can comprise any electronic device, such as a computer or tablet, including dedicated and may also include non-dedicated (proprietary and/or non-proprietary) applications. The host device can support secure and proprietary communication with the signal control unit 120 and can provide a user interface for the individual BCI user. In some variations, individuals use the host device 130 to control a wide variety of applications. In another variation, the receiver and transmitter function 102 and the signal control function 120 can be housed in the same hardware implanted in the individual.

This signal processing can include filtering, classifying, decoding, and transmitting the data received from the receiver and transmitter unit. In one variation, the inventive system simply comprises a signal control unit 120 and one or more external host devices 130, in which case the signal control unit 120 operates with a variety of systems. One benefit of using a dedicated signal control unit 120 is to provide a signal control unit 120 that allows for a power efficient low latency device for interaction with one or more electronic devices. In addition, the majority of the signal processing and data storage can occur in the signal control unit 120. The signal control unit 120 can be dedicated to signal processing and decision-making with custom applications to guide user interactions. In additional variations, the signal control unit 120 is configured to access a cloud-network 150 for computing and storage resources or for analytics. Offsetting such requirements from the implantable components allows for the minimization of the weight and size of the transmitter unit 120 and reduces the heat generated by the transmitter unit 102 during operation.

In addition, in some variations of the BCI system, moving all or most of the computing power to the signal control unit 120 allows any software updates to take place outside of the BCI user's body. In addition, this configuration allows the receiver and transmitter unit 102 to operate with lower power requirements to reduce the frequency of recharging. In alternate variations, the processing, storage, and communication functions can be divided between the receiver and transmitter unit 102, the signal control unit 120, and/or any external devices 130.

Generally, BCI system (the implant 100 and receiver and transmitter unit 102) captures motor intention from the brain (e.g., the motor cortex) and produces one or more electrical signals corresponding to the motor intention. Electrical signals can be captured from brain activity in regions other than the motor cortex. The signal control unit 120 decodes the electrical signals for utilization with a host device 130 for control of software applications (typically on the host device 130). In some cases, the host device 130 can be used to control additional digital devices (such as a computer, wheelchair, home automation systems, or other devices) that aid the individual 10 using the BCI.

Variations of the systems and methods described herein include a benefit of increasing longevity of the implanted components (e.g., 100, 104, 102) of the system to avoid repeated surgeries to replace implanted components. Accordingly, variations of the system and methods allow for a receiver and transmitter unit 102 that can be remotely and/or wirelessly recharged (e.g., capacitively) using an external power supply, as represented in FIG. 1B. Alternative variations can include a receiver and transmitter unit 102 that allows for a physical connection to an external power supply.

In one example, the system is designed for increased longevity as well as to provide increased mobility and autonomy for the BCI user 10. Such systems and methods can employ an architecture that distributes processing and data storage capabilities across non-implanted components of the system, with the implanted receiver and transmitter unit 102 responsible for obtaining and transmitting a signal indicative of the intent of the user. For example, the receiver and transmitter unit 102 can communicate with the electrode 100 such that when the electrode detects a brain signal from a brain of the BCI user 10, the receiver and transmitter unit is configured to transmit an electronic signal representative of the brain signal.

FIG. 1C illustrates a variation of a signal control unit 120 that houses a power supply 50, processor 52, and circuity 54-62 to enable wireless and/or wired electronic communication. The power supply 50 can comprise batteries or an external power source. The processor 52 is configured to apply one or more algorithms to decode the electronic signal from the transmission component. The signal control unit 120 can also be configured to produce an output signal upon determining that the electronic signal is representative of an intentional neural brain signal generated by the individual. Accordingly, the signal control unit 120 can house any number of communications circuitry or modules 54, 56, 58, 60, and 62 that can be used to electronically communicate with one or more external devices, as discussed below. The modules can provide the signal control unit 120 with wireless or wired communication capabilities. Examples of wireless communication include, but are not limited to, short-range wireless (e.g., blue tooth or blue tooth low energy), ultra-high frequency (such as low power device 433 MHz), as well as the ability to communicate via an HTTP/HTTPS using a WIFI or other network connection. In the variation illustrated in FIG. 1C, the signal control unit 120 can include one or more blue tooth low energy (BLE) modules to simultaneously or sequentially communicate with the receiver and transmitter unit 120 as well as any number of external devices 130, 132. The signal control unit 120 can also include different modules to simultaneously communicate with an alert device 128. Although not shown, the signal control unit 120 can include one or more speakers or alarms to play a tone, series of tones, pre-recorded message, or other audible message/sound. In an additional variation, one or more modules can comprise sensors to provide environmental and/or physiological information regarding the individual. Examples of environmental information can comprise the movement of the individual (using an accelerometer or GPS), ambient temperature, noise, etc. Examples of physiological data of the user can include heart rate, fatigue, caffeination, temperature, blood pressure, etc.)

In one variation of the system, the system is configured such that the signal control unit 120 is configured to interact with the receiver and transmitter unit 102 using a specific communication mode to limit the distribution of data from the receiver and transmitter unit 102. Such a specific communication mode can be encrypted, secured, and/or otherwise proprietary. This prevents transmission of data from the receiver and transmitter unit 102 to unauthorized devices. In some variations of the system, a host device is configured to receive data using this specific communication mode from the signal control unit 120. Therefore, one distinction between a host device 130 and other external devices 132 is that the external devices receive data using standard communication modes. In one variation of the system, the specific communication mode can comprise a proprietary and/or encrypted BLE, while communication with other external devices relies on other communication modes. This allows the signal control unit 120 to isolate/control various other communication modes to prevent inadvertent output of data (e.g., to prevent data transmitted unintentionally over the internet or to an external device).

In an additional variation of the system, the signal control unit 120 receives an electronic signal from the receiver and transmitter unit 102 and produces a decoded output signal. The signal control unit 120 is aware of which external devices are connected and active. Based on this information, the signal control unit 120 can produce an output signal for an HID. This HID output signal can be sent to the currently active end device. In one variation of the system, the signal control unit 120 more than external device can be connected to the signal control unit 120 but the signal control unit 120 is configured to only send the output signal to one device at a time. While the end devices can be paired in the host device 130 (e.g., by a caregiver), the user can control which device is active using neural signals. Pairing refers to the process that enables two electronic devices to establish a connection so they can communicate directly with each other, such as through Bluetooth wireless technology, and often includes verification steps to ensure the connection is secure. Additionally, during the active HID session with the end device, another active session with the host device can be ongoing using a distinct wireless protocol, which allows for secure input/output to/from the signal control unit 120 to the host device that informs the configuration and control of the signal control unit 120. Additionally, or alternatively, a third “proto-profile” or distinct wireless signal based on a third wireless protocol may utilize input and output signals communicating with the operating system of the host device (e.g., iOS Switch Control or Assistive Touch) to allow the individual to control the desktop and any apps on the host device or an “end” device instead of the host device based on context data from the host device. By utilizing a plurality of distinct wireless profiles (protocols or modes), the signal control unit 120 can communicate adaptively with a plurality of connected devices based on the decoded signal and the connected devices with which the signal control unit 120 is communicating.

FIG. 1D illustrates an example of a portable signal control unit 120 having a housing 168 with a minimal user interface on the housing to enable a lower power design as described herein. The housing 168 is selected to be portable (e.g., it can fit within the pocket of a garment or can be worn by the individual). The user interface is shown in FIG. 1D uses auditory, vibratory, and/or light feedback to provide user information. The housing 168 includes a power button 170 that can also toggle the signal control unit 120 between a locked and unlocked configuration as well as a sleep/low power mode. The power button 170 can be elastomeric or capacitive. The region 172 around the power button can provide general information. In the illustrated example, the information region 172 shows a ring-shaped light indicator to convey certain functions of the signal control unit 120. This light can vary in size and color and can pulsate to use varying frequencies of appearance to convey information. The housing 168 can also include a notification indicator 174 that can be used to call attention to system information such as errors or other notifications. The housing 168 can also include a power indicator 176 to show the power level/state of charge. Additionally, the housing 168 can include a connectivity indicator to convey the status of the signal control unit 120 connection with the receiver and transmitter unit or other component of the system. Variations of the signal control unit 120 can include any combination of the indicators described above. Moreover, in additional variations, the signal control unit 120 can include a detailed user interface rather than a limited user interface. Although not illustrated, the signal control unit 120 can include any additional ports, recovery buttons, speakers, etc.

The limited user interface with portable/small volume pocketable hardware provides a prosthetic hardware and functions to replace at least some lost mobility and function of the peripheral nervous system for the BCI user. To reduce power usage between user interactions host and end devices, the signal control unit 120 can be configured to disconnect any external device (e.g., host and/or external device) to save power when the receiver and transmitter unit 102 are in an idle mode. The signal control unit 120 can further leverage automation, or shortcuts, upon connection, to open the host device upon request of the signal control unit 120. The ability to monitor connected devices and selectively engage various communication modes can allow a signal control unit 120 to provide a BCI function for the user over a duration of at least 4, 8, 12, 24 hours on single battery charge, during which time the signal control unit 120 can receive, decode, and transmit distinct output signals to a plurality of external devices without external power. Accordingly, the signal control unit 120 can use information about the individual, including information about the components 130 that the individual interacts with, and use such information to improve decoding of the neural signal, as described below.

FIG. 1E illustrates another variation of a brain-computer interface (BCI) system used by individuals with mobility limitations to control peripheral devices such as personal electronic devices, internet of things (IoT) devices, or mobility vehicles or software applications running on such peripheral devices. An effective BCI system should allow the entire spectrum of individuals with mobility limitations to effectively control such peripheral devices or software applications, including those with severe mobility limitations, such as locked-in individuals. The individual controls a BCI system by regulating their brain activity, which is monitored by one or more components of the BCI system.

The BCI system can comprise a recording device or implant 100 (see FIG. 1F) and a computing device 130. Alternatively, as noted above, variations of the system can use a signal control unit. The recording device 100 can be configured to detect brain activity of the individual 10. The illustrated variation shows the recording device 100 100 is configured to be implanted within a brain vessel 104 of the individual as a stent 108 having multiple electrodes 103. The implant 100 can be implanted within a cortical or cerebral vein or sinus of the individual.

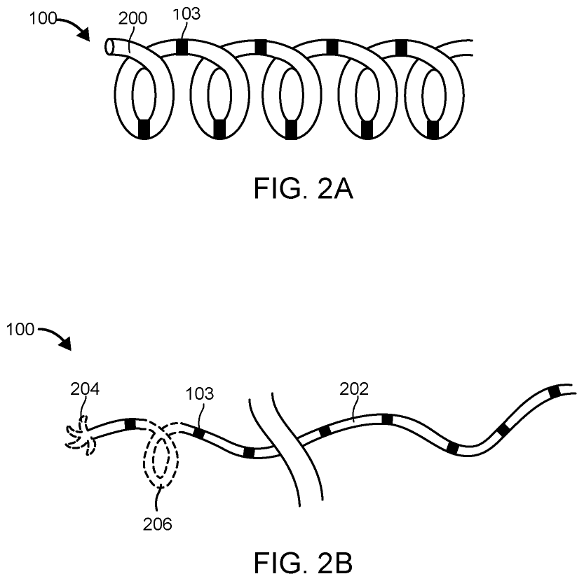

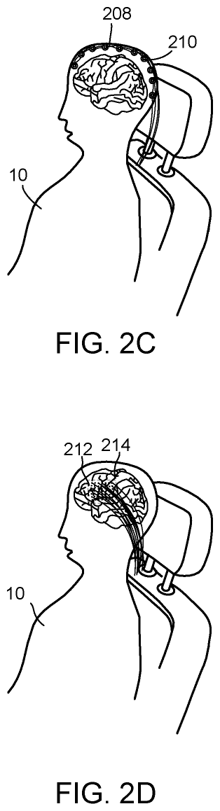

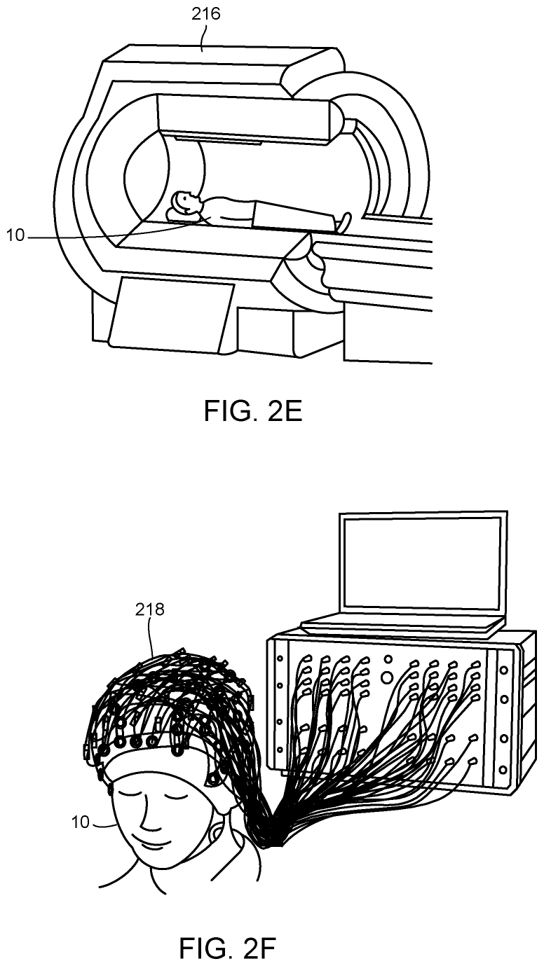

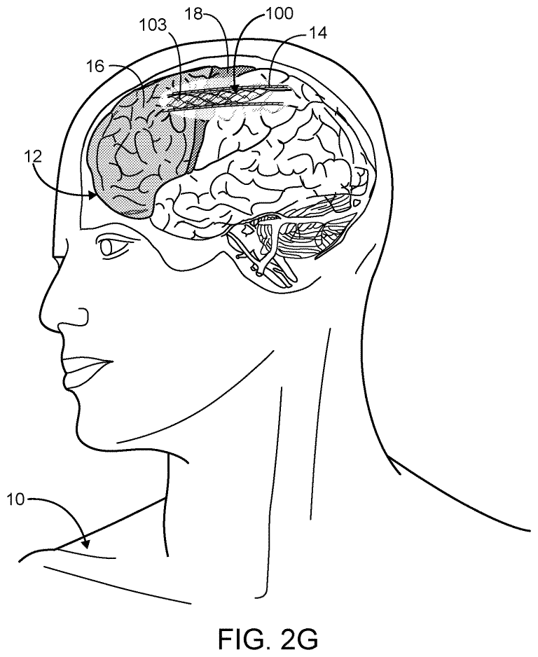

However, alternate configurations are within the scope of this disclosure, such as recording devices that are external to the body, as well as devices that are placed directly on or within brain tissue or placed on top of the brain tissue, under the dura. In other embodiments, the recording device 100 can be a non-invasive recording device 100 such as an electroencephalography (EEG) device (see, e.g., FIG. 2C), a functional magnetic resonance imaging (fMRI) device (see, e.g., FIG. 2E), or a functional near-infrared spectroscopy (fNIRS) device (see, e.g., FIG. 2F).

In other embodiments, the stent-electrode array 102 can be any of the stents, scaffolds, stent-electrodes, or stent-electrode arrays disclosed in U.S. Patent Pub. No. 2021/0365117; U.S. Patent Pub. No. 2021/0361950; U.S. Patent Pub. No. 2020/0363869; U.S. Patent Pub. No. 2020/0078195; U.S. Patent Pub. No. 2020/0016396; U.S. Patent Pub. No. 2019/0336748; U.S. Patent Pub. No. US 2014/0288667; U.S. Prov. No. 10,575,783; U.S. Pat. Nos. 10,485,968; 10,729,530, 10,512,555; U.S. Pat. App. No. 62/927,574 filed on Oct. 29, 2019; U.S. Pat. App. No. 62/932,906 filed on Nov. 8, 2019; U.S. Prov. App. No. 62/932,935 filed on Nov. 8, 2019; U.S. Prov. App. No. 62/935,901 filed on Nov. 15, 2019; U.S. Prov. App. No. 62/941,317 filed on Nov. 27, 2019; U.S. Prov. App. No. 62/950,629 filed on Dec. 19, 2019; U.S. Prov. App. No. 63/003,480 filed on Apr. 1, 2020; and U.S. Prov. App. No. 63/057,379 filed on Jul. 28, 2020, the contents of which are incorporated herein by reference in their entirety.

When the recording device 100 (e.g., the stent-electrode array 102) is implanted within a brain vessel 104 of the individual, each of the electrodes 103 of the recording device 100 can be configured to read or record the electrical activities of neurons within a vicinity of the electrode 103. The electrical activities of neurons are often recorded as rhythmic or repetitive patterns of activity that are also referred to as neural oscillations or brainwaves. Such neural oscillations or brainwaves can be further divided into bands by their frequency. For example, rhythmic neuronal activity between 14 Hz to 30 Hz is referred to as neuronal oscillations in a beta frequency range or beta-band.

When the 100 recording device (e.g., the stent-electrode array 100 of FIG. 1F) is implanted within a brain vessel 14 of the individual, the device 100 100 detects the neural oscillations of the individual, including any changes in such neural oscillations, over time in the beta-band (about 14 Hz to 30 Hz), alpha frequency range or alpha-band (about 7 Hz to 12 Hz), theta frequency range or theta-band (about 4 Hz to 7 Hz), gamma frequency range or gamma-band including a low-frequency gamma-band (about 30 Hz to 70 Hz) and a high-frequency gamma-band (about 70 Hz to 135 Hz), a delta frequency range or delta-band (about 0.1 Hz to 3 Hz), a mu frequency range or mu-band (about 7.5 Hz to 12.5 Hz), a sensorimotor rhythm (SMR) frequency range or SMR-band (about 12.5 Hz to 15.5 Hz), or a combination thereof. The device 100 can record changes in the power of such neural oscillations (e.g., as measured in decibels (dBs), micro-volts squared per Hz (μV2/Hz), average t-scores, average z-scores, etc.).

In some embodiments, the device 100 can be implanted within a cerebral or cortical vein or sinus of the individual or directly on or within brain tissue or placed on top of the brain tissue, under the dura. For example, the recording device 100 can be implanted within a superior sagittal sinus, an inferior sagittal sinus, a sigmoid sinus, a transverse sinus, a straight sinus, a superficial cerebral vein such as a vein of Labbe, a vein of Trolard, a Sylvian vein, a Rolandic vein, a deep cerebral vein such as a vein of Rosenthal, a vein of Galen, a superior thalamostriate vein, an inferior thalamostriate vein, or an internal cerebral vein, a central sulcal vein, a post-central sulcal vein, or a pre-central sulcal vein. In certain embodiments, the recording device 100 can be implanted within a vessel extending through a hippocampus or amygdala of the individual.

FIG. 1G illustrates that a communication conduit or lead 104 (e.g., a lead wire) can connect the recording device 100 with a receiver and transmitter unit 102 (FIG. 1H) that is communicatively coupled to the computing device 130 or a signal control unit as described above. Alternatively, the lead 104 can connect the recording device 100 directly with any external electronic device (e.g., a computing device 130).

The lead 104 can be a biocompatible wire or cable. When the device 100 is a stent-electrode array 102 deployed within a brain vessel (e.g., the superior sagittal sinus) 14 of the individual, the lead 104 can extend through one or more brain vessels and out through a wall of a vein of the individual. The lead 104 can be positioned under the skin of the individual to a region of the individual (e.g., beneath the pectoralis major muscle) where the receiver and transmitter unit receiver and transmitter 102 are implanted.

FIG. 1H illustrates a close-up view of an embodiment of a receiver and transmitter unit 102 receiver and transmitter. In some embodiments, the receiver and transmitter unit 102 can be configured to transmit signals received from the recording device 100 to the computing device 130 for processing and analysis. The receiver and transmitter unit 102 can also serve as a communication hub between the recording device 100 and the computing device 130. In certain embodiments, the computing device 130 can transmit commands or signals to the receiver and transmitter unit 102 to generate certain user outputs. Generating user outputs to train the individual to better control the BCI system 100 will be discussed in more detail in later sections.

In certain embodiments, the receiver and transmitter unit 102 can be an internal receiver and transmitter unit 102 implantable under the skin of the individual. For example, the receiver and transmitter unit 102 can be implanted within a pectoral region or within a subclavian space of the individual. In additional variations, an receiver and transmitter unit can be implanted in any location within the body (e.g., within a skull) or located entirely or partially outside of the body.

In other embodiments, the receiver and transmitter unit 102 can be an external receiver and transmitter unit 102 not implanted within the individual. In these embodiments, the lead 104 can extend through the skin of the individual to connect to the receiver and transmitter unit 102. In additional embodiments, the receiver and transmitter unit 102 can comprise both an implantable portion and an external portion.

In some embodiments, the receiver and transmitter unit 102 can transmit data or signals to the computing device 130 or receive data or commands from the computing device 130 via a wired connection. In other embodiments, the receiver and transmitter unit 102 can transmit data or signals to the computing device 130 or receive data or commands from the computing device 130 via a wireless communication protocol such as Bluetooth™, Bluetooth Low Energy (BLE), ZigBee™, WiFi, or a combination thereof and as described above.

FIG. 1I illustrates an additional variation of where a BCI (device 100, lead 104, and receiver and transmitter unit 102 coupled to an individual BCI user) is configured to use multiple and distinct communication channels of the signal control unit 120 to allow an individual BCI user to operatively engage one or more external devices to improve autonomy for the individual. The neural interface device 100 detects a brain signal or neural activity from the individual and is electrically coupled with a receiver and transmitter unit/component 102. Upon receiving a signal from the electrode component 100, the receiver and transmitter component transmits an electronic signal representative of the brain signal to the signal control unit 120. In one variation, the receiver and transmitter unit 102 transmits the electronic signal representative of the brain signal to the signal control unit 120 using a BLE transmission 22. As noted herein, the use of BLE allows for reduced power demands from the receiver and transmitter unit 102. However, additional variations of the systems and methods disclosed herein can use any wireless transmission modality as discussed herein. The system shown in FIG. 1I is further discussed in U.S. patent application Ser. No. 18/882,591, which is incorporated by reference in its entirety.

It is noted that FIG. 1I illustrates various specific wireless transmission modalities between the various components. For example, BLE 22, network/internet/HTTPS 24, UHF 26, short messaging service “SMS” 28. These specific wireless transmission modalities demonstrate one variation of an improved BCI device (100, 104, 102, 120, 130) operating in a much larger system. However, additional configurations of the system contemplate any wireless transmission modality being used between any components of the system.

The variation of the system shown in FIG. 1I includes a signal control unit 120 having a housing structure that is physically separate from the neural interface device (100, 102, 104) and is configured to be portable such that signal control unit 120 can remain with the individual while remaining in operative engagement the system. The signal control unit 120 processes the electronic signal representative of the brain signal using one or more processors that apply one or more algorithms to decode the electronic signal from the transmission component. As noted above, variations of the system include performing all or the majority processing of the electronic signal within the signal control unit 120. However, additional variations can include performing processing of the signal within one or more processors of the receiver and transmitter unit 102. Alternatively, or in combination, some processing can occur through external processing from a host device 130 or via one or more cloud-based networks 150.

Once the system that the electronic signal is representative of an intentional neural brain signal generated by the individual, the signal control unit 120 can transmit an output signal to one or more external devices using the signal control unit. In the illustrated variation, the signal control unit 120 exchanges data via a BLE transmission 22 with a personal host device 130 (such as a tablet computer, a computer, or other electronic device). However, additional variations of the system can include the signal control unit 120 directly communicating with other external electronic devices 160, 162, 164.

In some variations, the signal control unit 120 is configured to work with one or more end devices 132, where an end device is any digital device that supports HID profiles for a keyboard, mouse, or other peripheral devices. Interconnectivity with end devices in the individual's home can leverage traditional pass-key pairing to a BLE HID device with the help of the host device 130. In one example, an end device can include an eye-tracker or other applications that assist the individual in using the BCI interface, or advanced mixed-reality headsets that enable a “spatial computer” that merges digital content with the physical environment such as the Apple Vision Pro and Meta's Quest Pro and Quest 3.

Variations of the system include a host device 130 that supports secure and proprietary communication with the signal control unit 120 and provides the necessary user interface to support individual training and use of the entire BCI system. Individuals can use their host device to control a wide variety of applications, including but not limited to texting, writing documents, using social media, internet communications, shopping, interaction with home appliances and home automation, health applications, banking applications, etc.

The system shown in FIG. 1I also contemplates that one or more of the external electronic devices 130, 160, 162, 164 generate or pass data (“device data”). This device data is transmitted to the signal control unit 120, which can then transmit the device data back to any of the external electronic devices 130, 160, 162, 164.

FIG. 1I illustrates that the interface system can interact with cloud-based software and data through various communication pathways. In one variation, various host devices 130 can be synchronized with the cloud-based 150 data for a variety of purposes. The cloud network 150 can also store proprietary software relating to system components as well as authentication and certificate services, which are used both during product use and for production, manufacture, and installation of the system. The internet connection 24 (between the signal control unit 120 and cloud 150) can also be used to generate online notifications to a caregiver. In the example shown, the signal control unit 120 can send data to the cloud 150 that ultimately goes to a caregiver's cell phone 28 via SMS 154 messaging. Likewise, the caregiver can communicate with the user's host device 130 via SMS 154 or directly to the signal control unit 120 via the internet connection.

The system network and communication channels allow cloud connectivity for various individuals to monitor and review the performance of the BCI user to provide assistance or to improve the performance of the system. In some variations, the cloud network 150 stores neural data from the individual (or various other individuals) and can convey requests to third-party services, including support for notification use cases described in more detail elsewhere herein.

The cloud-network 150 also allows the system to access artificial intelligence (AI) that can be transmitted to any component of the system. In the example shown, the AI involves a large language model 152 that assists the BCI user to communicate with others by providing generative content as described in U.S. application Ser. No. 18/734,476 filed on Jun. 5, 2024, the entirety of which is incorporated by reference.

One additional benefit of the interface systems and methods described herein is that the low power, portability, and a number of distinct wireless communication modalities offer an “always-on” functionality. For example, given that the receiver and transmitter unit 102 is charged and the signal control unit 120 and host device 130 are powered, then the individual can use the complete system independently and on-demand for an extended period (e.g., 24 hours or more). This always-on feature allows the individual the ability to engage in digital daily living activities like telehealth, social media, communication, or adjusting interacting with various home interface systems that are based automation or control of appliances (e.g., 160, 162, 164). More importantly, the always-on feature can assist by providing potentially lifesaving messages to caregivers. By having the ability to run the system on battery power for an extended period allows this notification capability to work outside, away from home, and generally without the internet. Without an internet connection the system can still communicate to other devices, either in the individual's home (160, 162, 164) or to devices outside of the home. The signal control unit 120 can also communicate with devices if the host device 130 is offline. For example, the signal control unit 120 can send signals to one or more alert devices 128 using low power device 433 MHz. The always-on feature also allows the user to transition from an idle state and immediately request caregiver assistance (e.g. if the individual awakes at night and can instantly message a caregiver).

Another benefit of the system described includes selective control between a host 130 and any number of the external devices 160, 162, 164, where the control is adaptive based on detected bonding and/or active connections between the signal control unit 132 device and external devices 160, 162, 164 and based on differentiated intent information (confirmation of the intentional neural brain signal generated by the individual and decoded into an electronic signal transmitted to the SCU). An active connection means that the external device is bonded to the host 130 and active such that the external device is ready to exchange data with the host.

Adaptive control can occur with the host device 120 as well as any number of external devices selected by the user through the use of the signal control unit. The signal control unit 120, through bonding as discussed above, knows which devices are connected, and the signal control unit 120 translates the decoded intent signals generated by the individual adaptively based thereon. The term “bonding” is intended to refer to a relationship established between two devices, allowing for secure reconnection without re-pairing. This can be accomplished by the host device or any device in the system. In some cases, term pairing includes any process where devices exchange the information necessary to establish a connection or an encrypted connection.

As the signal control unit 120 translates the decoded signal into one or more output signals, the signal control unit 120 can use information on the number of bonded devices to take into account how to relay the output signal. This allows the BCI user individual autonomous control of interactions with a variety of different external devices without dependence on a caregiver. As one example, as noted above, if the individual generates an intent to contact a caregiver, once this intent is sent to the signal control unit for decoding and confirmation, the signal control unit 120 can generate an output command based on the bonded devices available. If the BCI user is in a situation where there are no bonded devices or if there is no network connectivity, then the signal control unit 120 can transmit the output through a fallback communication protocol (e.g., 433 MHz to an alert device 128). This capability can also be applied to system warnings.

In one variation, the BCI system can be configured to monitor the ability of the system to detect a brain signal from the user and determine that the electronic signal associated is representative of an intentional neural brain signal generated by the individual. In some cases, an individual might suffer from deteriorating health such that the signals generated by the brain change or deteriorate, causing a BCI system that once was properly functioning to no longer function due to the deteriorating condition of the individual. In such a case, the signal control unit 120 can be configured to provide notice to a caregiver or other medical practitioner.

In an additional variation of the system shown in FIG. 1I, the signal control unit 120 receives an electronic signal from the receiver and transmitter unit 102 and produces a decoded output signal. The signal control unit 120 is aware of which external devices are connected and active. Based on this information, the signal control unit 120 can produce an output signal for an HID. This HID output signal can be sent to the currently active end device. In one variation of the system, the signal control unit 120 more than external device can be connected to the signal control unit 120 but the signal control unit 120 is configured to only send the output signal to one device at a time. While the end devices can be paired in the host device 130 (e.g., by a caregiver), the user can control which device is active using neural signals. Additionally, during the active HID session with the end device, another active session with the host device can be ongoing using a distinct wireless protocol, which allows for secure input/output to/from the signal control unit 120 to the host device that informs the configuration and control of the signal control unit 120. Additionally or alternatively, a third “proto-profile” or distinct wireless signal based on a third wireless protocol may utilize input and output signals communicating with the operating system of the host device (e.g., iOS Switch Control or Assistive Touch) to allow the individual to control the desktop and any apps on the host device or an “end” device instead of the host device based on context data from the host device. By utilizing a plurality of distinct wireless profiles (protocols or modes), the signal control unit 120 can communicate adaptively with a plurality of connected devices based on the decoded signal and the connected devices with which the signal control unit 120 is communicating.

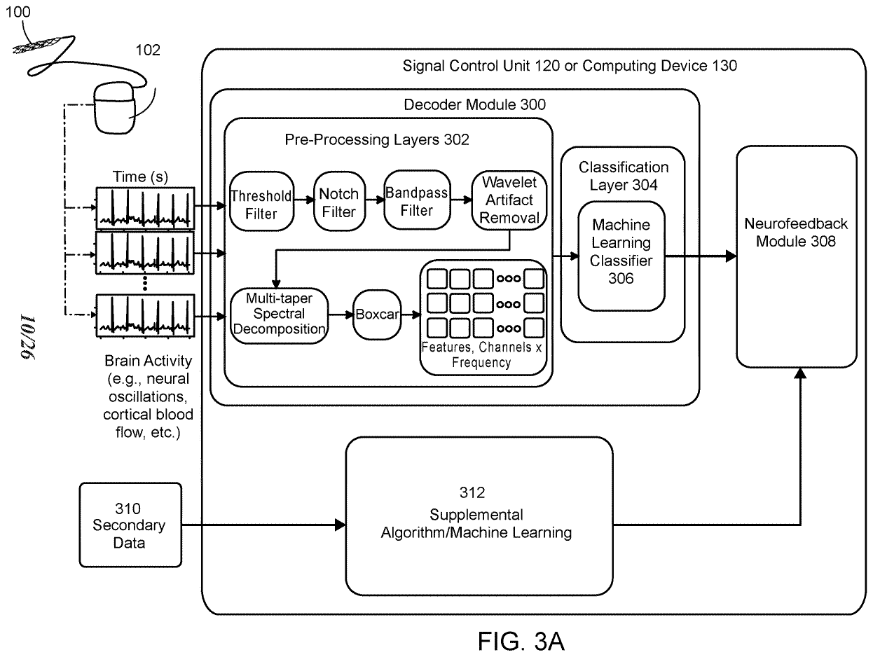

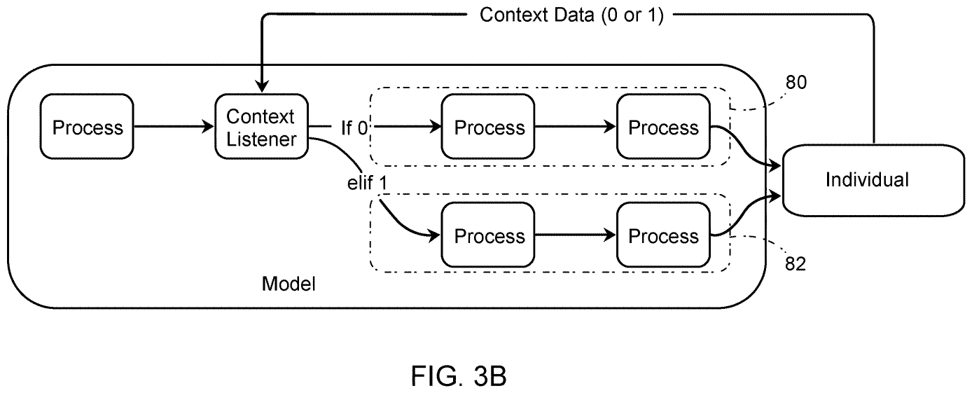

In some variations of the system, a connection between a signal control unit 120 and a host device 130 is immediately disconnected after data transfer and when data is not being transferred between the signal control unit 120 and host device 130 to preserve power. However, this can increase the latency of the system. In order to improve the user experience for the systems described in this disclosure, e.g., as shown in FIGS. 3A and 3B, the components of the system can be configured to minimize system latency so that the user can use brain activity to engage with host/external devices. For example, both the host and end devices can keep their connections active when the signal control unit 120 is in use. This can allow a low system latency (e.g., less than 100 ms) from detection of the neural signal, transmission and wireless receipt of the signal by the signal control unit 120, decoding and translation of neural signal to an output signal using the signal control unit 120, and transmission of a second wireless transmission from the signal control unit 120 to one or more external devices. The selective control between host and end devices can be adaptive based on detected bonds (between the signal control unit 120 and external devices) and differentiated intent information (decoded signals from the neural signals received from the electrode device and decoded by the signal control unit 120 device).

FIG. 1I provides just one illustration of the variety of electronic devices and systems that allow for interaction by the individual user of the BCI. As discussed above, the implant 100, signal control unit 102, and receiver and transmitter unit 102 are either implanted or with the individual. Accordingly, the signal control unit 120 can monitor the individual for physiological data, data related to actions taken by the individual when accessing one or more devices, or by monitoring the types of devices that the individual can access using the system. Accordingly, as discussed below, the decoding algorithms that are used to translate the neural activity to an actionable electronic command can dynamically and adaptively change to produce different or altered outputs based on this monitoring data/factors related to the individual. Such contextualized decoding is intended to better assist the individual when using the BCI and allows for the application of a decoding algorithm that is best suited to the activity that the individual is undertaking. For example, such factors can include the selection of certain decoding algorithms based on the type of electronic device that is either engaged with the system or the electronic device that the individual is presently using. Such data can be generated by the external devices (e.g., 130, 148, 160, 162, 164) or by a processor (e.g., in the signal control unit 120). Or by additional sensors that monitor the individual.

FIG. 2A illustrates another variation of a recording device 100 as a coiled wire 200 comprising a plurality of electrodes 103. The coiled wire 200 can serve as the endovascular carrier for the electrodes 103 and can be used in vessels that are too small to accommodate the stent-electrode array 102. The coiled wire 200 can be a biocompatible wire or microwire configured to wind itself into a coiled pattern or a substantially helical pattern. The electrodes 103 can be arranged such that the electrodes 103 are scattered along a length of the coiled wire 200. More specifically, the electrodes 103 can be affixed, secured, or otherwise coupled to distinct points along a length of the coiled wire 200.

The electrodes 103 can be separated from one another such that no two electrodes 103 are within a predetermined separation distance (e.g., at least 10 μm, at least 100 μm, or at least 1.0 mm) from one another. In some embodiments, the wire 200 can be configured to automatically wind itself into a coiled configuration (e.g., helical pattern) when the wire 200 is deployed out of a delivery catheter. For example, the coiled wire 200 can automatically attain its coiled configuration via shape memory when the delivery catheter or sheath is retracted. The coiled configuration or shape can be a preset or shape memory shape of the wire 200 prior to the wire 200 being introduced into a delivery catheter. The preset or pre-trained shape can be made to be larger than the diameter of the anticipated deployment or implantation vessel to enable the radial force exerted by the coils to secure or position the coiled wire 200 in place within the deployment or implantation vessel.

The wire 200 can be made in part of a shape-memory alloy, a shape-memory polymer, or a combination thereof. For example, wire 200 can be made in part of Nitinol (e.g., Nitinol wire). The wire 200 can also be made in part of stainless steel, gold, platinum, nickel, titanium, tungsten, aluminum, nickel-chromium alloy, gold-palladium-rhodium alloy, chromium-nickel-molybdenum alloy, iridium, rhodium, or a combination thereof.

FIG. 2B illustrates yet another variation of the recording device 100 as an anchored wire 202 comprising a plurality of electrodes 103. The anchored wire 202 can serve as the endovascular carrier for the electrodes 103 and can be used in vessels that are too small to accommodate either the coiled wire 200 or the stent-electrode array 102. The anchored wire 202 can comprise a biocompatible wire or microwire attached or otherwise coupled to an anchor or another type of endovascular securement mechanism. FIG. 2B illustrates that the anchored wire 202 can comprise a barbed anchor 204, a radially-expandable anchor 206, or a combination thereof (both the barbed anchor 204 and the radially-expandable anchor 206 are shown in broken or phantom lines in FIG. 2B). In some embodiments, the barbed anchor 204 can be positioned at a distal end of the anchored wire 202. In other embodiments, the barbed anchor 204 can be positioned along one or more sides of the wire or microwire. The barbs of the barbed anchor 204 can secure or moor the anchored wire 202 to an implantation site within the individual. The radially-expandable anchor 206 can be a segment of the wire or microwire shaped as a coil or loop. The coil or loop can be sized to allow the coil or loop to conform to a vessel lumen and to expand against a lumen wall to secure the anchored wire 202 to an implantation site within the vessel. For example, the coil or loop can be sized to be larger than the diameter of the anticipated deployment or implantation vessel to enable the radial force exerted by the coil or loop to secure or position the anchored wire 202 in place within the deployment or implantation vessel.

The electrodes 103 of the anchored wire 202 can be scattered along a length of the anchored wire 202. More specifically, the electrodes 103 can be affixed, secured, or otherwise coupled to distinct points along a length of the anchored wire 202. The electrodes 103 can be separated from one another such that no two electrodes 103 are within a predetermined separation distance (e.g., at least 10 μm, at least 100 μm, or at least 1.0 mm) from one another. Although FIG. 2B illustrates the anchored wire 202 having only one barbed anchor 204 and one radially-expandable anchor 206. It is contemplated by this disclosure that the anchored wire 202 can comprise a plurality of barbed anchors 204 and/or radially-expandable anchors 206.

FIG. 2C illustrates another variation of a recording device 100 that can be a non-invasive device such as an electroencephalogram (EEG) device 208. The EEG device 208 can be a head-mounted EEG apparatus. For example, the EEG device 208 can be an EEG cap or an EEG-visor configured to be worn by the individual. The EEG device 208 can comprise a plurality of non-invasive electrodes 210 configured to be in contact with the scalp of the individual.

The brain activity detected by the EEG device 208 can be neural oscillations or brainwaves of the individual, similar to those recorded by the recording device 100. For example, the EEG device 208 can record neural oscillations, including any changes in such neural oscillations, over time in the beta-band (about 14 Hz to 30 Hz), alpha frequency range, or alpha-band (about 7 Hz to 12 Hz), theta frequency range or theta-band (about 4 Hz to 7 Hz), gamma frequency range or gamma-band including a low frequency gamma-band (about 30 Hz to 70 Hz) and a high frequency gamma-band (about 70 Hz to 135 Hz), a delta frequency range or delta-band (about 0.1 Hz to 3 Hz), a mu frequency range or mu-band (about 7.5 Hz to 12.5 Hz), a sensorimotor rhythm (SMR) frequency range or SMR-band (about 12.5 Hz to 15.5 Hz), or a combination thereof. The EEG device 208 can record changes in the power of such neural oscillations (e.g., as measured in decibels (dBs), micro-volts squared per Hz (μV2/Hz), average t-scores, average z-scores, etc.).

FIG. 2D illustrates that in yet another embodiment of the system 100, the recording device 100 can be an electrocorticography (ECoG) device 212 (also referred to as an intracranial EEG device). The ECoG device 10 can be a flexible or stretchable electrode mesh or one or more electrode patches implanted or placed on a surface of the brain of the individual. The electrode-mesh or electrode patch can comprise a plurality of electrodes 214 arranged on the mesh or patch, respectively.

The brain activity detected by the ECoG device 212 can be neural oscillations or brainwaves of the individual 14, similar to those recorded by the stent-electrode array 102. For example, the ECoG device 212 can record neural oscillations, including any changes in such neural oscillations, over time in the beta-band (about 14 Hz to 30 Hz), alpha frequency range, or alpha-band (about 7 Hz to 12 Hz), theta frequency range or theta-band (about 4 Hz to 7 Hz), gamma frequency range or gamma-band including a low-frequency gamma-band (about 30 Hz to 70 Hz) and a high-frequency gamma-band (about 70 Hz to 135 Hz), a delta frequency range or delta-band (about 0.1 Hz to 3 Hz), a mu frequency range or mu-band (about 7.5 Hz to 12.5 Hz), a sensorimotor rhythm (SMR) frequency range or SMR-band (about 12.5 Hz to 15.5 Hz), or a combination thereof. The ECoG device 212 can record changes in the power of such neural oscillations (e.g., as measured in decibels (dBs), micro-volts squared per Hz (μV2/Hz), average t-scores, average z-scores, etc.).

FIG. 2E illustrates another variation of the recording device that uses a functional magnetic resonance imaging (fMRI) machine 216. The fMRI machine 216 can detect changes in blood flow and blood-oxygen levels within the brain of the individual as the individual 14 engages in neural activity as part of the neurofeedback training. Such changes in blood flow and blood-oxygen levels are the indirect consequence of the individual's neural activity.

In some embodiments, the fMRI machine 216 can measure the brain activity of the individual using blood-oxygen-level dependent (BOLD) contrast imaging. For example, the brain activity of the individual 14 can be expressed as changes in the BOLD signal. In other embodiments, the fMRI machine 216 can measure the brain activity of the individual using arterial spin labeling (ASL) rather than BOLD contrast imaging.

FIG. 2F illustrates that in another embodiment of the system 100, the recording device can be a functional near-infrared spectroscopy (fNIRS) device 218. The fNIRS device 218 can use near-infrared light (NIR) to measure hemodynamic activity in the brain of the individual as the individual engages in neural activity as part of the neurofeedback training. For example, the fNIRS device 218 can comprise a fNIRS cap configured to be worn on the head of the individual. The fNIRS device 218 can comprise a plurality of NIR light sources and detectors (called optodes). The fNIRS device 218 can measure the hemodynamic activity by measuring changes in oxy-hemoglobin concentrations (HbO) and deoxy-hemoglobin (HbR) concentrations in the cerebral cortex.

FIG. 2G shows a variation in which electrodes 103 of the device 100 are positioned within a vessel 14 of a brain 12 of an individual 10. As noted above, the device 100 can be coupled a BCI. Placement of the device 100 within a vessel 14 allows for monitoring neural activity in deep regions of the brain 12 as well as across different regions. In this variation, the device is placed across several regions, including the primary motor cortex 18 and the frontal cortex 16. As noted below, such a configuration allows for the BCI to obtain data of neural activity across the supplementary motor cortex, which is involved in inhibition, error monitoring, and decision making, as well as the frontal cortex, which is involved in high order cognitive processes. As discussed below, such a configuration allows the BCI to obtain electrical signals associated with neural activity from one region of the brain and to obtain secondary data from a different region of the brain.

In an additional variation, the device 100 can be in the superior sagittal sinus (SSS), which provides direct neural recordings from cortical midline structures, including the dorsal anterior cingulate cortex (dACC), pre-supplementary motor area (pre-SMA), and medial prefrontal cortex (mPFC)—key regions implicated in error processing. Recording brain activity in such regions allows for the BCI to capture secondary data from these regions and presents an opportunity for passive cognitive error detection into BCI applications, allowing for real-time system self-correction and enhanced user interaction without requiring explicit behavioral action.

In additional variations, BCI systems can use an electrode device positioned in other regions of the brain than those shown in FIG. 2G. Moreover, BCI systems can use multiple electrode devices, or a combination of devices as described above, to monitor neural activity to produce an output signal as well as to obtain secondary data as described below.

FIG. 3A illustrates an example where the device 100 records or detects neural activity such that the receiver and transmitter unit 102 or the signal control unit produces an electronic signal representing the neural activity and transmits the electronic signal(s) to one or more computing devices/computer processors. For example, the computer processor can comprise the signal control unit 120 or can comprise an external electronic computing device 130. These devices 120/130 can be programmed to convert brain activity into predictions concerning an intention of the individual. As will be discussed in more detail in later sections, the intention can be a thought conjured by the individual or an attempt made by the individual to move a body part of the individual (e.g., move a left hand or left ankle of the individual). Moreover, the intention can also be a thought conjured by the individual or an attempt made by the individual to reach or maintain a neural rest state. In these cases, the intention is not directly related to the individual focusing their attention or viewing certain graphics rendered on a display of a device such as the computing device.

FIG. 3A illustrates one example of software layers or modules running on the computing device 120/130 of the BCI system. For example, one or more processors of the computing device 120/130 can be programmed, e.g., using one or more algorithms, to execute software instructions, making up the various software layers or modules. In other embodiments, not shown in the figures but contemplated by this disclosure, any references to a computing device or computer processor can also refer to a control unit or controller embedded within the receiver and transmitter unit 102. In further embodiments contemplated by this disclosure, any references to the computing device can also refer to a computing device or control unit/controller that is part of the recording device 100 (for example, when the recording device 100 is an fMRI machine or an fNIRS device).

The pre-processing layer 302 can comprise a plurality of software filters or filtering modules configured to filter and smooth out the raw signals obtained from the recording device 100. For example, when the recording device 100 is an endovascular recording device configured to be implanted within a brain vessel of the individual (e.g., the stent-electrode array 102), the brain activity of the individual can be monitored using the various electrodes 103 of the recording device 100. As a more specific example, the brain activity of the individual can be sampled every 100 ms such that 100 ms “chunks” or bins of the raw neural signals recorded can be passed to the pre-processing layer 302 for processing and smoothing.

The pre-processing layer 302 can first apply a (1) threshold filter to filter out the raw signals using certain thresholds. The pre-processing layer 302 can then apply a (2) notch filter to perform, for example, 50 Hz notch filtering, and also apply a (3) bandpass filter to perform, for example, 4-30 Hz Butterworth bandpass filtering. The pre-processing layer 302 can then apply a (4) wavelet artifact removal filter to perform wavelet-based artifact rejection, a (5) multi-taper spectral decomposition filter to perform multi-taper spectral decomposition, and a (6) boxcar smoothing filter to perform temporal boxcar smoothing. The filtered data can then be fed to the classification layer 304 of the decoder module 300.

As shown in FIG. 3A, the computing device can comprise at least one decoder module 300 and a neurofeedback module 308 for generation and/or transmission of an output signal produced by the decoder 302.. The decoder module 300 can further comprise one or more pre-processing layers 302 and a classification layer 304. As discussed below, the decoder module can select different decoder settings based upon the individual BCI user's environmental or application factors.

In another variation, the systems and methods described herein allow a BCI system to automatically or selectively use different decoder settings based on one or more factors. Such factors can include, but are not limited to, environmental factors or application factors.

Some decoders are more suitable in some circumstances than others. BCI decoders are typically used to change the state of a computer application. Some state transitions have higher potential functional and social consequences than others (e.g., pressing send on an email compared to typing a character). It may be appropriate to change decoders based on their comparative accuracy/speed and the current context of the application state. However, changing decoders can be unwieldy and can undermine autonomy for an individual to have to switch decoders manually.

The application software, via a commercially available platform, including a mobile device platform, can detect a variety of information, including what type of application is being used and about where and how the patient is using the system. This “context” may include the activity in which the user is engaging such as typing, using social media, engaging with a “tiled” graphic user interface, browsing through an internet browser, interacting with a mixed-reality headset; navigating a map, watching a video, engaging in a computer. game. The context can also include environmental factors like temperature, lighting, ambient noise etc.; external physiological data like heart rate available as part of the Apple health ecosystem or other similar systems.

Given the context, the system can adjust its decoding settings based on this information or a variety of information. Such an adjustment can include changing the algorithm, changing the underlying neural phenomena being used by the decoder, or both.

Different algorithms have different characteristics. For example, asynchronous switch algorithms make predictions on a quasi-continuous basis (e.g., one prediction every 100 ms). This contrasts with synchronous switch algorithms, which make predictions on timescales that users can aptly respond to (e.g., 2-4 seconds). Therefore, asynchronous algorithms tend to have a higher likelihood of producing false positives than synchronous algorithms. A contextually aware system may wish to change to a more accurate yet slower synchronous decoder when potentially eliciting high-consequence application state changes (e.g., sending an email).

FIG. 3B illustrates a basic example of using context to determine which decoder 80, 82 that is used to decode a signal in a BCI. In this example, the individual is monitored to obtain contextual data regarding the individual's behavior, condition, or situation. This contextual data can include any information that will be relevant to the individual. For example, such data include information on which electronic devices are presently connected to the BCI system (e.g., see FIG. 1I), which device is currently being used by the individual, what is the screen/GUI featuring: whether a keyboard or the tile format or something else, data on whether the system locked or in use, data on whether one or more components of the BCI system and electronic devices are powered down or powered up, receiver and transmitter battery status, accelerometer data to determine if the individual is moving or stationary, what end device is being used by the host device (connectivity information), physiological data of the user (e.g., heart rate, fatigue, caffeination, temperature, blood pressure, etc.)

The context data acts as an input to determine which decoding algorithm or process (e.g., 80 or 82) is used to produce an output for the individual to use to engage the BCI system and/or associated electronic devices. While the illustration in FIG. 3B shows two decoding choices 80 82. The system can include any number of decoding algorithms or processes.

In practice, a neural interface device 100 is configured to detect a brain activity of an individual. A component, such as the receiver and transmitter unit or the signal control unit, generates and transmits an electronic signal to a computer processor (e.g., See FIGS. 1E and 1I) the signal control unit 120, an external computer 130, etc.) The system, typically the computer processor, also monitors the individual to generate contextual data. Alternatively, or in combination, the contextual data can be generated by the electronic device(s) that the individual is using, physiological data associated with the individual, and/or specific actions taken by the individual while engaging the electronic devices. The electronic signal is then processed using the computer processor to produce an output signal, where the computer processor is configured to selectively apply at least one algorithm from a plurality of algorithms for decoding the electronic signal, wherein selection of the at least one algorithm is at least partially dependent on the contextual data. This output signal is then electronically transmitted to one or more external electronic devices such that the individual is able to interact with the one or more external electronic devices using the brain activity (e.g., see FIG. 1I transmission 22, 24, 26, etc.)

Generating the contextual data can occur by actively monitoring the individual prior to or during interaction between the individual and the one or more external electronic devices. As shown in FIG. 1I, the electronic devices can provide contextual data, which includes information associated with electronic devices (e.g., the type of device, the type of control required, etc.).

Additionally, different neural phenomena also have different characteristics. This may be in terms of signal-to-noise ratio, timescale of manifestation, and context of action. For example, oscillatory bursts are volitionally produced and fast-acting, so are suitable for applications such as typing. Error-related potentials (ErrPs), on the other hand, are not volitionally produced but can indicate when an erroneous action has been taken [1]. Switching the decoder to look for ErrPs after application state changes may be a beneficial undertaking.

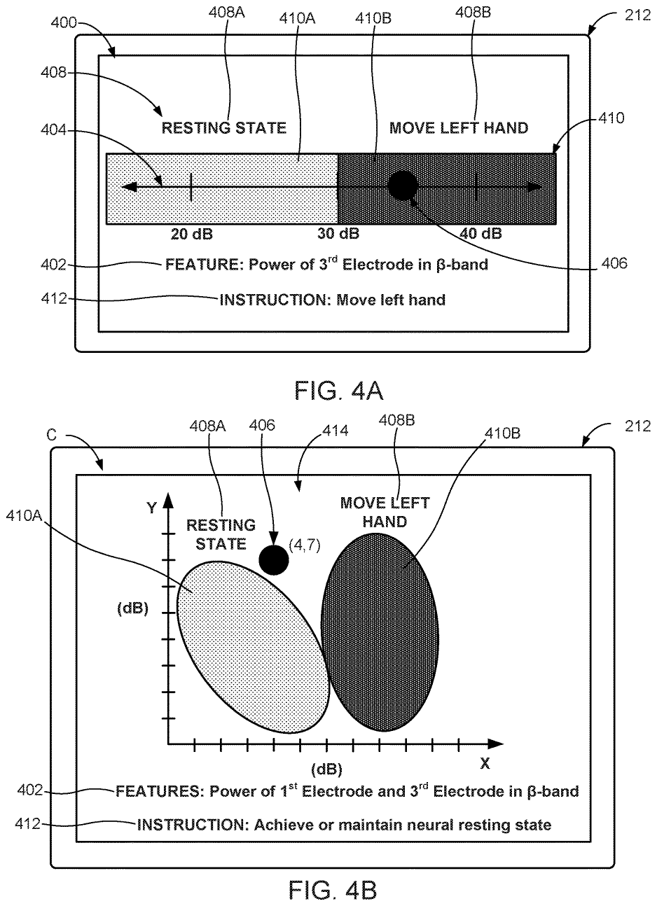

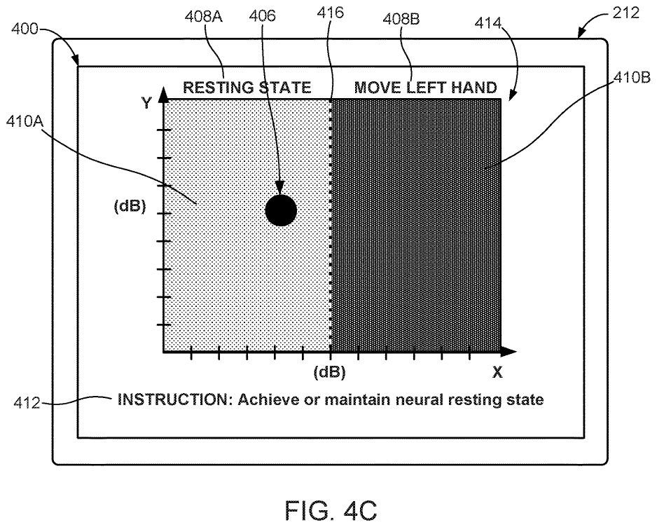

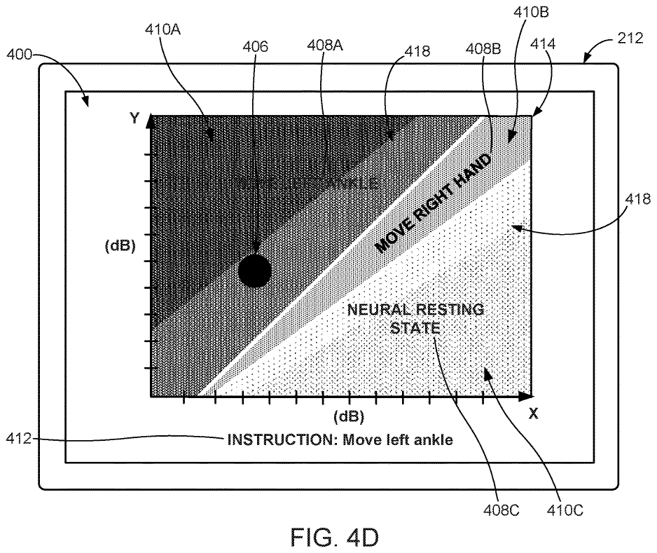

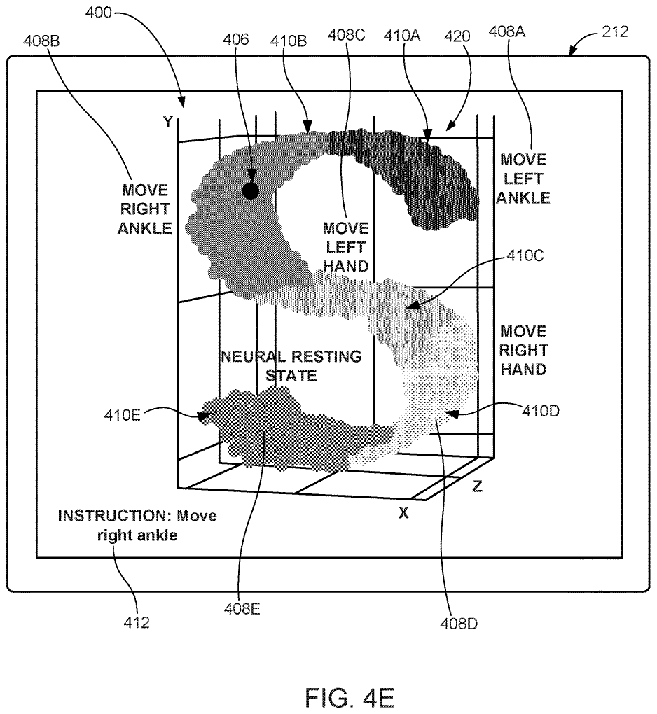

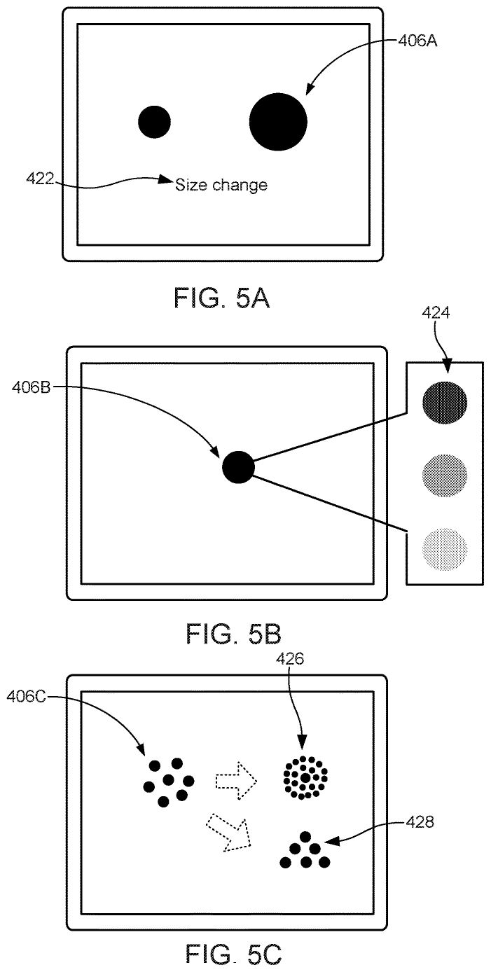

In some embodiments, a neurofeedback graphical user interface (GUI) 400, as shown in FIGS. 4A-4E can be displayed on a display 212 communicatively coupled to the computing device or processor. The individual can view the neurofeedback GUI 400 on the display 212 while the individual is undergoing neurofeedback training. As will be discussed in more detail in later sections, a moveable graphic element 406 (see e.g., FIGS. 4A-4E and 5A-5C) can be shown on the neurofeedback GUI 400 representing a current brain activity of the individual recorded by the recording device 100. The neurofeedback GUI 400 and the graphic element 406 can be constructed in a way that reduces the complexity of the brain activity recorded by the recording device 100 into a form that is engaging and easy to comprehend by the individual. Moreover, the neurofeedback GUI 400 and the graphic element 406 can aid the individual in producing brain activity that aligns with a desired intention of the individual.

The computing device can convert brain activity recorded by the recording device 100 into predictions concerning the intention 408 of the individual by being trained to map or associate previously recorded brain activity to certain intentions 408. For example, the computing device can be trained using training set data gathered from the individual as the individual repeatedly initiates, sustains, and terminates certain intentions 408. During these training sessions, the brain activity of the individual can be recorded by the recording device 100.

Once the computing device is trained or calibrated using training set data gathered from the individual, the computing device can control certain peripheral devices or software applications running on such peripheral devices based on the predicted intentions 408 of the individual. For example, the computing device can be communicatively coupled to (i.e., in wired or wireless communication with) a peripheral device such as a personal electronic device, an IoT device, a mobility vehicle or a software application running on the peripheral device. The computing device can transmit signals or commands to the peripheral device or the software application to control the operation or functionality of the peripheral device or the software application in response to the predicted intentions 408 of the individual. For example, the computing device can instruct a mobility vehicle (e.g., a wheelchair) transporting the individual to move in a forward direction in response to the individual formulating or carrying out an intention 408 to move the individual's left hand.

However, as previously discussed, whether the individual is able to use the BCI system to successfully control the peripheral device or software application depends on the ability of the individual to self-regulate their brain activity and to consistently produce brain activity calibrated to the intention 408. Therefore, neurofeedback training can improve the individual's control over the BCI system 100 and, ultimately, improve the individual's control over one or more peripheral devices communicatively coupled to the BCI system 100 or software application running on such peripheral devices.

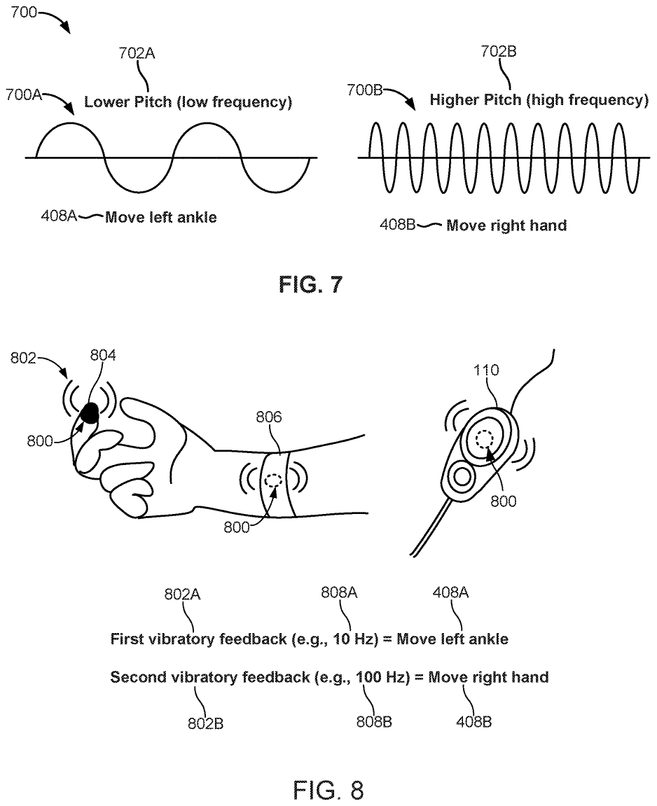

The classification layer 304 can comprise one or more machine learning algorithms or classifiers 306 to classify the resulting data segments or bins into an intention 408 (see FIGS. 4A-4E and 6-8) of the individual. In some embodiments, the machine learning algorithm or classifier 306 can be a supervised learning model such as a support vector machine (SVM). In other embodiments, the machine learning algorithm or classifier can be a Gaussian mixture model classifier, a Naïve Bayes classifier, or another type of machine learning classifier.

The classification layer 304 can be trained or calibrated to classify or make predictions concerning the intention 408 of the individual based on previously recorded brain activity. For example, the classification layer 304 can predict the individual's intentions 408 several times per second. The classification layer 304 can be trained using training data collected from the individual.

In some embodiments, the training phase can involve the individual repeatedly initiating, sustaining, and terminating certain thoughts or attempting certain actions while the individual's brain activity is recorded by the device 100. For example, one such training session can involve the individual repeatedly resting for 5 seconds, followed by attempting to move their left hand for 5 seconds. The individual's brain activity during this training session can be recorded, and the recorded brain activity can be mapped to the individual's intentions 408 to rest and move their left hand, respectively.

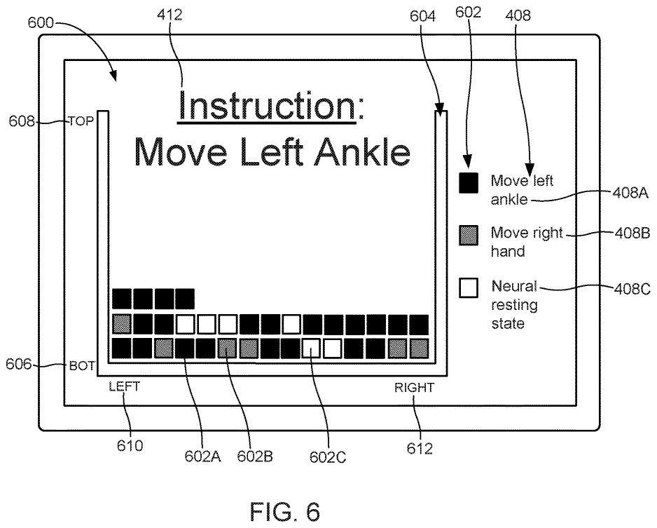

As shown in FIG. 3A, the classification layer 304 can feed the predicted intention 408 of the individual to the neurofeedback module 308. In some embodiments, the neurofeedback module 308 can be configured to construct certain neurofeedback GUIs (see, e.g., FIGS. 4A-4E and 6) to be displayed to the individual via a display 212 communicatively coupled to the computing device to aid the individual in producing or recreating brain activity that aligns with a desired intention of the individual. In other embodiments, the neurofeedback module 308 can also transmit commands or signals to a user output device (e.g., a speaker or a tactile feedback component) communicatively coupled to the computing device to generate a user output (e.g., sounds or tactile feedback) to aid the individual in producing brain activity that aligns with a desired intention of the individual. The neurofeedback module 308 will be discussed in more detail in the following sections.

FIG. 3A also shows the use of secondary data 310 that is transmitted to a processing component of the BCI (e.g., the signal control unit 120 or other computing device) such that the processor applies one or more supplemental algorithms or machine learning processes 312 to either determine whether to alter the output signal generated by the decoder module 300 or to alter an effect of that output signal.

As discussed below, the secondary data can comprise neural data recorded from the same region of the brain used to decode the output signal. Alternatively, or in combination, the secondary data can comprise neural data recorded from different regions of the brain. Alternatively, the secondary data can be any data associated with the individual, such as muscle movement, breathing, heart rate, etc. In some variations of the method, the secondary data is obtained contemporaneously with the neural activity or shortly after generation of the output signal. For example, the supplemental algorithm can be developed by monitoring an individual during use of the BCI. In those cases where the individual incorrectly or unintentionally generates an output signal, data associated with the individual's reaction to a false positive can be recorded for use to detect an error in future use of the system. Alternatively, the algorithm and models can be trained to identify secondary data associated with the individual when the individual produces neural activity that correctly/intentionally generates an output signal.

The use of the supplemental algorithm 312 that processes secondary data 310 can be used to alter the output signal prior to transmitting the output signal to an electronic device. For example, if the secondary algorithm predicts that the output signal was generated in error, the algorithm can prevent the output signal from being transmitted to the electronic device or various other components of the BCI. Alternatively, or in combination, the supplemental algorithm can be used to further instruct the electronic device to ignore the output signal based on the prediction that it was generated in error. In the latter case, the algorithm makes a determination to alter the effect of the output signal on the electronic device.

As noted above, because the secondary data is data associated with the individual while generating the neural activity to produce an output signal, the secondary data can be measured contemporaneously with the initial neural activity or shortly after generation of the output signal. For example, the system assesses the secondary data that was produced during the initial neural activity or shortly thereafter. In some variations, analysis of the secondary data occurs on data that was recorded at the time of the neural activity or at the time the BCI produced the output signal.

The use of secondary data allows for methods and systems of detecting covert cognitive error signals via an endovascular BCI and leveraging these signals for adaptive error correction in real-time BCI applications. In one example, the methods allow for identification of covert error-related neural signals from midline cortical regions using a neural interface device, which obtains local field potential recordings. Variations of the method include the use of machine learning classifiers to differentiate error-related potentials (ERN, Pe) and associated high-gamma activity from background noise.

The methods can further include predictive text correction based on covert recognition of typing errors, detecting discrepancies between intended and decoded speech before user correction. In addition, the methods and systems can include autocorrecting movement trajectories based on error awareness before explicit correction.

The methods and systems can integrate with artificial intelligence by enhancing AI-mediated interactions through embedding real-time covert error feedback loops.

In one example, the neural interface device is implanted in the SSS and records local field potential activity from the dACC, SMA, and prefrontal regions. Error-related signals are extracted based on event-related potentials (ERN, Pe) and high-gamma power changes (70-150 Hz).

The systems and method can further include data processing and machine learning models. Including: time-lock error signals to motor or cognitive tasks (e.g., typing, imagined speech); use of deep learning classifiers (CNNs/RNNs) or state-space models to predict covert error signals; implementation of real-time decoding for dynamic system adjustments.

The systems and methods allow for additional BCI applications and adaptive interfaces. The covert error detection can be integrated into brain-to-device interaction and can use real-time AI-assisted autocorrection. The systems and methods can be expanded to non-motor cognitive interfaces, such as AI-assisted reasoning and decision-making.

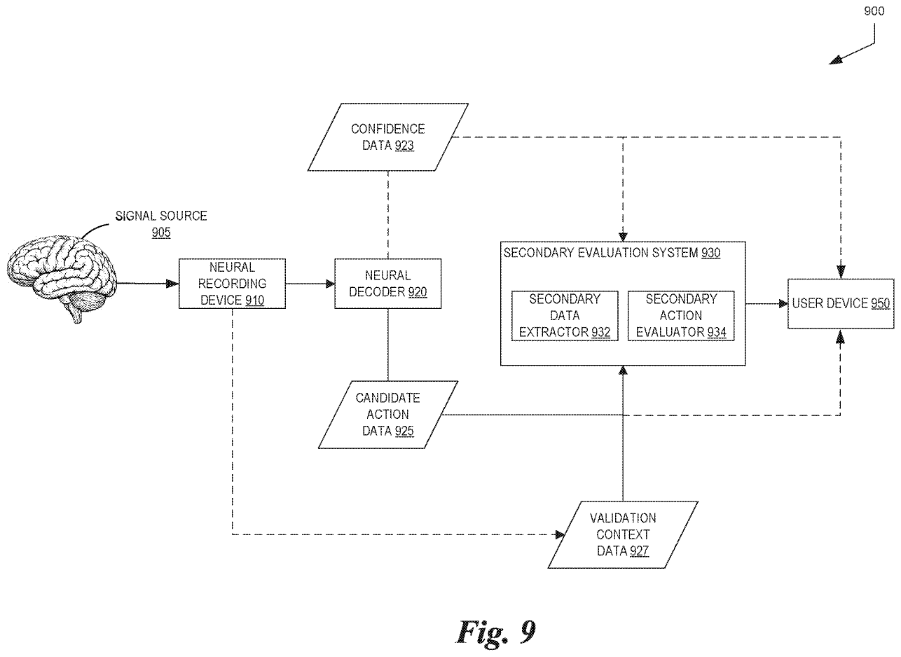

With reference to an illustrative example, FIG. 9 shows an example environment 900 for providing secondary evaluation of a candidate action based on candidate action data and validation context data using a secondary evaluation system. The environment 900 includes a signal source 905, a neural recording device 910, a neural decoder 920, a secondary evaluation system 930, and a user device 950.

The signal source 905 is at least a portion of a brain of a user of the user device 950. Neural signal information may be generated by the neural recording device 910 based on information generated by monitoring the signal source 905. The neural recording device 910 may be any type of recording device (e.g., the recording device 100, signal control unit 120, receiver and transmitter unit 102, etc.) described above herein with respect to FIGS. 1A-2G and used to generate information based on the activity of a human brain.

The neural decoder 920 receives neural signal data from the neural recording device 910 and generates candidate action data 925 based on decoding the neural signal data. The neural decoder 920 may include a plurality of neural decoders 920. The neural decoder 920 may be implemented as a multi-layer perceptron with a softmax output layer over a set of discrete output classes, a recurrent neural network such as a long short-term memory network or gated recurrent unit, a transformer decoder, a temporal convolutional network, or any combination of the preceding options. Where a plurality of neural decoders 920 are used, individual neural decoders 920 may receive neural signal data generated from distinct portions of the signal source 905 by the neural recording device 910 and/or by different neural recording devices. In some embodiments, a neural decoder 920 may classify the neural signal data as one of a plurality of actions. A neural decoder 920 may generate candidate action data 925 that indicates a most likely action from among a set of potential intentions available to that neural decoder 920.

The neural decoder 920 may, in some embodiments, generate confidence data 923. The confidence data 923 may be associated with an individual action represented in the candidate action data 925. Where the neural decoder 920 generates an indication of a most likely action associated with the neural signal data from among a plurality of available actions, the confidence data 923 may include a confidence associated with two or more of the available actions. The neural decoder 920 may provide the candidate action data 925 directly to the user device 950. The neural decoder 920 may provide the candidate action data 925 to the secondary evaluation system 930 to allow the secondary evaluation system 930 to evaluate the candidate action data 925 in view of validation context data 927 before action data is provided to the user device 950.

The candidate action data 925 describes an action to be performed by the user device 950 based on processing neural signals at a neural decoder 920. The candidate action data 925 may be formatted according to a BCI human interface device (HID) report used by the user device 950. The candidate action data 925 may be associated with confidence data 923 as described below herein. The candidate action data 925 may indicate a user has formed an intent to activate a switch, move a cursor, type using an onscreen keyboard, enter text directly, actuate a motor, or implement any other action performable by the user device 950 and controlled by the user.

The confidence data 923 may include a confidence value generated by a neural decoder 920. For example, the neural decoder 920 may implement one or more machine learning models to decode neural data received from the signal source 905. Some or all of the machine learning models may generate a confidence value associated with the output of the respective machine learning model. The confidence value may be the confidence data 923, or the confidence value generated by multiple machine learning models may be combined to generate the confidence data 923. When multiple confidence values are combined, the confidence values may be weighted (e.g., based on previous knowledge about the accuracy of the machine learning models implemented by a neural decoder 920). The confidence data 923 may be structured to provide each confidence value in addition to, or instead of, individual confidence values. The confidence data 923 may include an identifier used to associate the confidence data 923 with a specific candidate action data 925 generated by the neural decoder 920.

The validation context data 927 may include current, historical, or predicted information associated with the user device 950, a user's physiological state, a user's behavior, a user's neural signal data (e.g., generated by the neural recording device 910 and provided to the secondary evaluation system 930 separately from the candidate action data 925 generated by the neural decoder 920), a contextual risk identifier, an environment in which the secondary evaluation system 930 or user device 950 is operating, an environment where the user is located, or any other information available to the secondary evaluation system 930. The validation context data 927 may be received by the secondary evaluation system 930 independently from any neural signal data or candidate action data 925 received by the secondary evaluation system 930 from the neural recording device 910 or neural decoder 920. The secondary evaluation system 930 may analyze the validation context data 927 to determine whether to alter (e.g., to delay, suppress, or modify) candidate action data 925 associated with validation context data 927 or based on comparing the candidate action data 925 to validation context data 927. The validation context data 927 may be data generated within a window of time surrounding the generation or intended execution of candidate action data 925. For example, the secondary evaluation system 930 may use validation context data 927 generated within a threshold time before and after receiving the candidate action data 925 to determine whether the action indicated by the candidate action data 925 is valid or an error. In some cases, the secondary evaluation system 930 may cause display in the user interface of a request for confirmation of an action defined in candidate action data 925 based on the validation context data 927 before causing execution of the action.

The validation context data 927 may in some embodiments be generated by a machine learning model. For example, the validation context data 927 may be generated by a neural foundation model trained to generate brain state information (e.g., as a latent representation of a brain based on neural signal data) for a user. The validation context data 927 generated by the neural foundation model may indicate an error state (e.g., an unintended action, an intention to reverse a previously indicated action, etc.) based on the user's neural data. Further, the validation context data 927 generated by the neural foundation model may include a recommendation to address a determined error state. For example, the validation context data 927 from a neural foundation model may indicate that the secondary action evaluator 934 should generate modified action data to modify, suppress, repeat, or otherwise alter candidate action data 925.

A contextual risk identifier may include information indicating a risk or relative importance of an action being performed based on the candidate action data 925. The risk or relative importance of the action being performed may relate to a potential danger (e.g., physical, reputational, psychological, etc.) from an action being performed in error by the user device 950. For example, where user device 950 is a robotic arm, and the user is controlling movements of a robotic arm in a physical environment, a contextual risk identifier may indicate a high associated risk. The secondary action evaluator 934 may then evaluate the candidate action data 925 based on the risk associated with controlling the robotic arm. In another example, the user device 950 may be a tablet computing device, and the user may be controlling an onscreen keyboard of the user device 950 to send a text message. The contextual risk identifier in this example may indicate a lower associated risk, and the secondary action evaluator 934 may analyze the candidate action data 925 based on this lower associated risk. The contextual risk identifier may also be used, in part, to determine a latency window between when the neural decoder 920 generates candidate action data 925 based on a motor intent a time when the user device 950 executes the associated action.

The secondary evaluation system 930 may buffer or otherwise temporarily store the validation context data 927 for a fixed period of time. For example, the secondary evaluation system 930 may store the validation context data 927 generated at a first time until a threshold second time has passed. The threshold second time may be based at least in part on a latency window during which a candidate action in the candidate action data 925 may be modified or suppressed by the secondary evaluation system 930. Advantageously, temporary storage of the validation context data 927 may reduce the total amount of storage resources needed by the secondary evaluation system 930 by allowing validation context data 927 that may no longer impact use of the user device 950 or generation of a modified action by the secondary action evaluator 934.

Examples of physiological state information include: heart rate, heart rate variability (HRV), skin conductance, galvanic skin response, respiratory rate, pupil dilation, blood pressure, EMG from residual muscle activation, micro-movements, facial tension, jaw clenching, postural shifts, head movement, blink rate, reaction latency, head nodding patterns, time-of-day data, session duration, and eye tracking instability. The physiological state information may be used to determine at least: elevated stress, sudden arousal spike, calm baseline, physiological instability, muscle activity inconsistent with decoded intent, co-contraction or tension, stable posture aligned with task, fatigue, reduced vigilance, prolonged session duration.

For example, determined physiological conditions of elevated stress, a sudden spike in arousal, physiological instability, muscle activity inconsistent with a decoded intent, co-contraction or tension, fatigue, reduced vigilance, or prolonged session duration, may cause the secondary evaluation system 930 to suppress, modify, delay, or otherwise alter an action in the candidate action data 925 associated with the physiological state to allow for additional processing of neural signal data.

Examples of behavioral data associated with a current user, or another user, that may be used by the secondary evaluation system 930 include, but are not limited to: commands frequently reversed, rapid undo sequences, high correction frequency for specific actions, repeated cancellations, preferred command sequences, typical response timing, common interaction pathways, habitual behaviors, history of confirmation gestures, timing between intent and correction, frequency of manual overrides. The secondary evaluation system 930 may use such data to determine whether to delay, suppress, modify, or otherwise alter candidate action data 925 from the neural decoder 920. For example, an action known to be prone to errors, a pattern of reversal associated with an action, repeated undo requests, actions determined to be inconsistent with historical behavior, deviations from a pattern of typical user behavior, a pattern of activity suggesting hesitation or uncertainty by the user, or a history of rapid overrides may be determined by the secondary evaluation system 930 from the examples of validation context data 927 above and the secondary evaluation system 930 may delay, modify, suppress, or otherwise alter a candidate action data 925 associated with such a determination.

Examples of environmental context data that may be used by the secondary evaluation system 930 to determine whether to suppress, modify, delay, or otherwise alter an action associated with candidate action data 925 include, but are not limited to: whether a location associated with the user, the neural decoder 920, or the user device 950 is a home, private, or public location; whether a location associated with the user, the neural decoder 920, or the user device 950 is a known safe environment or an unfamiliar environment; whether the user, the neural decoder 920, or the user device 950 is stationary or in motion; geofenced zones; noise level; lighting level; temperature; whether a location associated with the user, the neural decoder 920, or the user device 950 is crowded or isolated (e.g., based on determining a likely number of people within a surrounding area); if a user is driving, conversing, or multitasking; or whether the validation context data 927 indicates use of an assistive device, or engagement with another application or device. For example, if the user is in motion, then the secondary evaluation system 930 may modify the candidate action data 925 to reduce a magnitude of an action indicated by the candidate action data 925. In another example, the secondary evaluation system 930 may modify or suppress candidate action data 925 (e.g., to delay or prevent execution of an action) when the validation context data 927 indicates a user is in an unfamiliar environment, in an environment with a high noise level or that is otherwise distracting, in an environment with adverse conditions (e.g., cold, hot, crowded, etc.), or when the user is under a high cognitive load (e.g., based on determining the user is multitasking).

Examples of language context data that may be used by the secondary evaluation system 930 to determine whether to suppress, modify, delay, or otherwise alter an action associated with candidate action data 925 include, but are not limited to: semantic coherence; grammatical matches/mismatches; consistency of word sequencing; or a determined improbability of the use of a word or words within the context of nearby words, a sentence, sentences, a paragraph, or paragraphs, or a document or collection of documents. For example, if a word or phrase generated based on user input (such as through a virtual keyboard) is determined by the signal control unit 120 to be implausible alone or in the context of other language information, the signal control unit 120 may modify or suppress the related candidate action data 925. In a further example, where a word or phrase appears inconsistent with related language based on context the secondary evaluation system 930 may suppress the candidate action data 925 to delay execution of the indicated action. In another example, where the secondary evaluation system 930 determines that there is a strong semantic alignment and the confidence data 923 indicates a low confidence, the secondary evaluation system 930 may cause the action described by the candidate action data 925 to be executed by the user device 950.

Examples of motion or trajectory context data that may be used for secondary evaluation by the secondary evaluation system 930 to determine whether to suppress, modify, delay, or otherwise alter an action associated with candidate action data 925 include, but are not limited to: proximity of a user to an obstacle; an indication of a collision risk; or location information indicating a user is in an unsafe movement zone. For example, if a user is determined based on the validation context data 927 to be at risk of a collision within a time period (e.g., based on a threshold time), then the secondary evaluation system 930 may suppress candidate action data 925 (e.g., until a collision risk has passed). In another example, if a user takes a minor deviation from an expected motion path, the secondary evaluation system 930 may cause the user device 950 to delay execution of the action to allow for additional time to confirm the action associated with the validation context data 927 is intended. In a further example, the secondary evaluation system 930 may determine that the user is moving along a clear path based on the validation context data 927, and even where confidence data 923 generated by the neural decoder 920 indicates a low confidence may cause the user device 950 to execute an action indicated by the candidate action data 925.

Altering an action or instruction indicated in the candidate action data 925 is referred to throughout the present disclosure. Altering an action or instruction, as used herein, may refer to at least updating candidate action data 925 to confirm that an action or instruction in the candidate action data 925 should be executed, modifying a switch assignment or activated switch indicated by the candidate action data 925, delaying execution of the candidate action data 925 by the user device 950 (e.g., by providing a length of time before which an action indicated in the candidate action data 925 should not be executed), suppressing execution of the candidate action data 925 by the user device 950 (e.g., by sending an instruction to the user device 950 to ignore a previously received candidate action data 925), transmitting a non-action to the user device 950 (e.g., to cause the user device 950 to update a neurofeedback display, to cause the user device 950 to wait for a further instruction, etc.), preventing transmission of the candidate action data 925 to the user device 950 (e.g., where the secondary evaluation system 930 transmits confirmed candidate action data 925 to the user device 950 determining to not transmit the validation context data 927, instructing the neural decoder 920 not to transmit the candidate action data 925 to the secondary evaluation system 930, etc.), transmitting an instruction to the user device 950 to ignore received candidate action data 925 (e.g., based on a specific identifier associated with the candidate action data 925, using a duration of time in which the user device 950 should ignore all candidate action data 925 received by the user device 950, etc.).

The secondary evaluation system 930 is a computing device that evaluates candidate action data 925 based on validation context data 927 to provide information (e.g., an instruction, an action, an indication of a switch activation, etc.) to a user device 950. The secondary evaluation system 930 includes a secondary data extractor 932, and a secondary action evaluator 934. While the secondary evaluation system 930 is described as one computing device herein for clarity, elements of the secondary evaluation system 930 (e.g., the secondary data extractor 932 or secondary action evaluator 934), or functionality performed by the secondary evaluation system 930 or elements of the secondary evaluation system 930 may be performed by a cloud-based system. For example, the validation context data 927 may be received at a cloud-based system and the secondary data extractor 932 may extract relevant data before providing the extracted relevant data to the secondary action evaluator 934.

The secondary data extractor 932 parses validation context data 927 received by the secondary data extractor 932 to identify information related to a candidate action data 925. To determine information in the validation context data 927 is relevant to a candidate action data 925, the secondary data extractor 932 may determine an action type (e.g., based on a description or HID protocol definition in the candidate action data 925), switch type, whether an indicated action is continuous (e.g., controlling a cursor) or discrete (e.g., selecting a button or activating a mouse click), from the candidate action data 925. Further, the secondary data extractor 932 may determine whether validation context data 927 is relevant to a candidate action data 925 based on the type of validation context data 927 (e.g., physiological information, location information, time information, etc.), a time at which the validation context data 927 is received (e.g., within a threshold time of when candidate action data 925 is generated or received), a device type from which the validation context data 927 is received, or any other information identifiable from the validation context data 927. The secondary data extractor 932 may generate a link between candidate action data 925 and associated validation context data 927. For example, the secondary data extractor 932 may generate a structured data object including at least a portion of candidate action data 925 and associated information from the validation context data 927.

The secondary action evaluator 934 evaluates the candidate action data 925 in the context of associated validation context data 927 to determine whether candidate action data 925 is to be modified, suppressed, delayed, executed, or otherwise altered. The secondary action evaluator 934 may further alter the candidate action data 925 based on the associated validation context data 927, or generate and transmit a signal to the user device 950, to implement the alteration.

The secondary action evaluator 934 may implement one or more machine models trained to evaluate received validation context data 927 in the context of candidate action data 925 to determine whether the candidate action data 925 is an intended action or an error. A machine learning model implemented by the secondary action evaluator 934 may be trained on a training data set including information on correctly-generated action data and action data associated with a false action (e.g., generated in error). The trained machine learning model may act as a classifier, generating a classification output based on the candidate action data 925 and the validation context data 927 to indicate whether the candidate action data 925 is intended or an error. The classification output of a machine learning model of the secondary action evaluator 934 may include a confidence value representing a confidence of the machine learning model that the classification is correct. The confidence value may be normalized (e.g., between 0 and 1).

Training and applying a machine learning model as described here to classify candidate action data 925 as intended or an error presents several advantages. Initially, generation of the training data set may be automated. For example, the secondary evaluation system 930, or another system, may maintain a history of actions and an indication as to whether the action was reversed or otherwise undone. The indication of whether the action was reversed or not may act as the classification label for each action in the historical action data. The labelled historical action data may then be associated with validation context data 927 generated within a time window (e.g., a fixed time before and after the action data was generated) around the action data generation or execution of an action based on the action data. Therefore, generation and labelling of the historical action data used to train the machine learning model may be simplified and automated, reducing the need for human input and reducing misclassifications in the training data.

In some embodiments, the secondary action evaluator 934 may implement a neural foundation model based on a machine learning model trained to generate or analyze a brain state (e.g., a latent representation of neural information). The secondary action evaluator 934 may implement a pre-trained and/or post-trained neural foundation model, a detector head coupled to a pre-trained backbone, or another classifier configured to infer one or more cognitive or control-related states, including error awareness (e.g., by the user from which the neural recording device 910 records neural data), hesitation, conflict detection, corrective planning, behavioral inhibition, or an unintended-action state. Based on the inferred state, the secondary action evaluator 934 may generate altered or additional action data that may cause the user device 950 to permit, delay, modify, attenuate, suppress, cancel, or replace the candidate action data 925 with a non-action.

Further, in some embodiments the secondary action evaluator 934 may not operate continuously. Because the secondary action evaluator 934 in some embodiments is configured as a classifier to differentiate between intended or mistaken (e.g., generated in error) candidate action data 925, the secondary action evaluator 934 may be executed when candidate action data 925 is received by the secondary evaluation system 930 but otherwise may not be executed. Advantageously, executing the secondary action evaluator 934 only when candidate action data 925 is received reduces the computer resources required by the secondary evaluation system 930 while improving the accuracy of action data executed by the user device 950.

In further embodiments, the secondary action evaluator 934 may implement a rules-based approach to determine whether to alter the candidate action data 925. The secondary action evaluator 934 may store or access a set of associations between types of validation context data 927, or information included in validation context data 927, and alterations to be made to related candidate action data 925. For example, the validation context data 927 may include information on keystrokes a user is entering into an onscreen keyboard of the user device 950. The secondary action evaluator 934 may compare the keystroke information to a dictionary in the user's language to determine whether the keystroke information is likely to form a valid word in the language. The secondary action evaluator 934 in this example may then modify the candidate action data 925 when the keystroke information indicates the word is not valid based on the comparison so that the altered candidate action data 925 causes entry of a valid word in the onscreen keyboard of the user device 950.

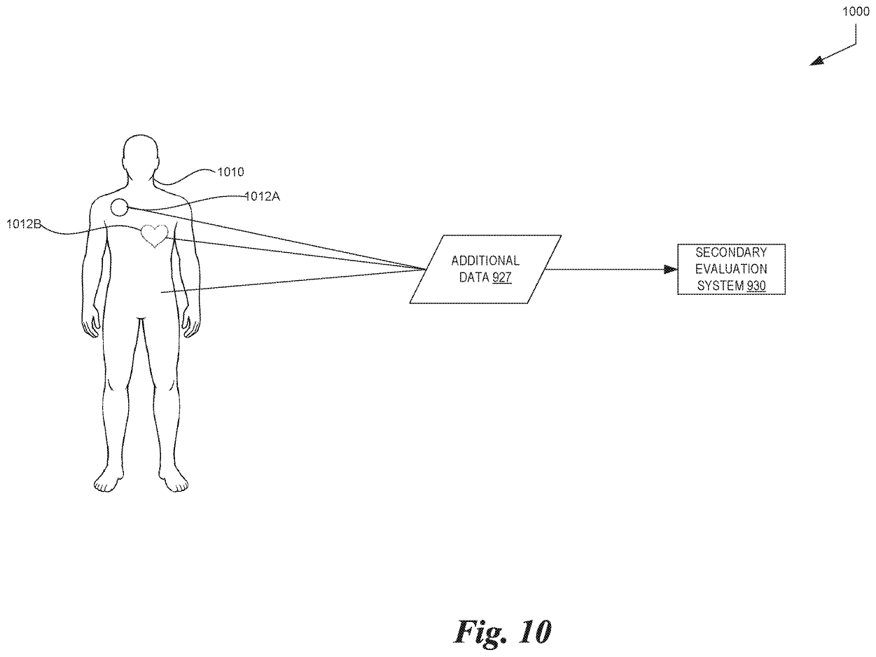

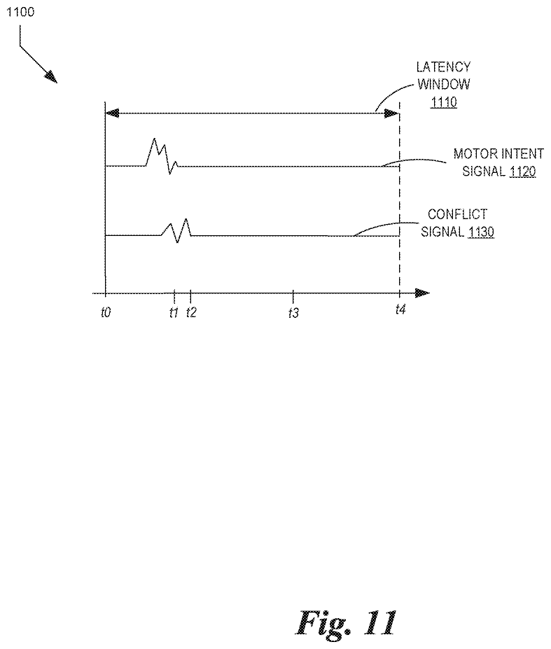

FIG. 10 shows an example environment 1000 for acquiring some types of validation context data 927 used to evaluate a candidate action using the secondary evaluation system 930. The environment 1000 includes a user 1010 from whom the validation context data 927 is acquired by monitoring devices 1012A-1012B, and the secondary evaluation system 930 that processes the acquired validation context data 927. As shown in this example environment 1000, one or more monitoring devices may be connected to, or arranged near, the user 1010 to allow for the acquisition or generation of validation context data 927 for the secondary evaluation system 930. For example, the monitoring device 1012A may generate motion-related information for the user 1010 (e.g., via an inertial measurement unit, accelerometer, GPS module, etc.). In another example, the monitoring device 1012B may generate heart rate information, or other physiological information, for the user (e.g., the physiological information described above herein with respect to FIG. 9).

The information generated by the monitoring device 1012A and monitoring device 1012B may be individually provided to the secondary evaluation system 930 as validation context data 927 (e.g., as a data stream or in bursts from a monitoring device). The information generated by the monitoring device 1012A and monitoring device 1012B may be combined (e.g., at fixed intervals or when new information is received from a monitoring device) to generate the validation context data 927 (e.g., as a structured data object in a defined format). The validation context data 927 is then received by the secondary evaluation system 930 for additional processing (e.g., parsing by the secondary data extractor 932).

FIG. 11 shows an illustrative chart showing a latency window in which a conflict signal may be associated with a motor intent signal. The visualization shows a latency window 1110, motor intent signal 1120, and a conflict signal 1130 at different points in time as represented by the x-axis of the graph representation 1100.

The latency window 1110 is a time within which a motor intent signal 1120 (e.g., as represented in candidate action data 925) may be analyzed to alter an action. The latency window 1110 may be a fixed length of time. For example, the latency window 1110 may be 10 ms, 50 ms, 100 ms, 200 ms, 500 ms, 1 s, or any other length of time within which candidate action data 925 may be altered by the secondary evaluation system 930. The latency window 1110 may be dynamic and may be adjusted based on an action type associated with the motor intent signal 1120. For example, where the motor intent signal 1120 is associated with candidate action data 925 that would move a robotic arm, the latency window 1110 may be longer as the latency between intent and action may be less important to the user. In another example, the latency window 1110 may be set to a shorter time when the user is actively typing a text message, as the latency will be more noticeable between keystroke entries to the user and may negatively impact the accuracy of the user's typing.

The latency window 1110 may begin at t0 on the graph representation 1100 within a threshold time of when a user begins to form a motor intent associated with a motor intent signal 1120. The time t1 may indicate when the neural decoder 920 has decoded the user's motor intent to generate candidate action data 925. The time t2 may indicate when a conflict associated with a motor intent signal 1120 has been identified in validation context data 927 by the secondary evaluation system 930 (e.g., the secondary data extractor 932) and the secondary data extractor 932 has generated a conflict signal associated with the conflict. Time t3 is a time after the conflict has been identified by the secondary evaluation system 930, but before the time t4 where time t4 is the end of the latency window 1110. Time t3 may be a time at which the secondary evaluation system 930 modifies, suppresses, or otherwise alters candidate action data 925 associated with the motor intent signal 1120.

In some embodiments, the latency window 1110 may be associated with an update cycle (e.g., a rate at which the user device 950 updates based on candidate action data 925) of the user device 950. The update cycle may represent a time between the recording of a motor intent signal 1120 (e.g., by the neural recording device 910) and the user device 950 executing an action based on the candidate action data 925 associated with that recorded motor intent signal 1120. The update cycle may include the time at which a conflict signal 1130 is received by the secondary evaluation system 930, although in some cases the conflict signal 1130 may be received before the motor intent signal 1120 with which the conflict signal 1130 is associated. The update cycle may also include processing time for the motor intent signal 1120 (e.g., by the neural decoder 920). The length of time of the update cycle may be the same or different from the length of time of the latency window 1110. An update cycle may occur in a fixed length of time (e.g., 5 ms, 10 ms, 25 ms, 50 ms, 100 ms, 200 ms, 500 ms, 1 s, etc.). The length of time for an update cycle may be dynamic, and may vary based on the type of user device 950, type of action indicated by the candidate action data 925, a number of actions being processed within a time window, or any other information available to the neural decoder 920, secondary evaluation system 930, or user device 950.

The motor intent signal 1120 is signal information generated by the signal source 905, neural recording device 910, or neural decoder 920, associated with a motor intent of the user. The motor intent signal 1120 may be a signal generated by the signal source 905 directly from a measurement of a portion of a user's brain. The motor intent signal 1120 may be a derived signal (e.g., filtered or otherwise processed) generated by the neural recording device 910 for transmission to a neural decoder 920. The motor intent signal 1120 may be the output signal of a neural decoder 920 that indicates variance in the strength of a user's motor intent over time. As used herein, a motor intent refers to a user's formation of an intent in their mind to perform a motor action (e.g., squeezing a fist, moving a hand, moving a limb, etc.).

The conflict signal 1130 is a signal generated or extracted by the secondary evaluation system 930 based on validation context data 927 received by the secondary evaluation system 930. The conflict signal may indicate a magnitude of an event associated with the validation context data 927 and a time at which the event occurred. For example, if the user is in motion, the conflict signal 1130 may indicate a magnitude of such motion of the user over time. In another example, if the user is determined to be distracted, the conflict signal 1130 may indicate a magnitude of the user's distraction over time as determined from the validation context data 927 by the secondary evaluation system 930. The conflict signal 1130 may be generated based on neural information from the user. For example, specific neural signatures such as error-related negativity, error positivity, high-gamma activity associated with a user recognizing a mistake in an action that is being performed or was previously performed, or other neural activity from a region of the user's brain associated with cognitive control (e.g., frontal lobe regions) may be the conflict signal 1130.

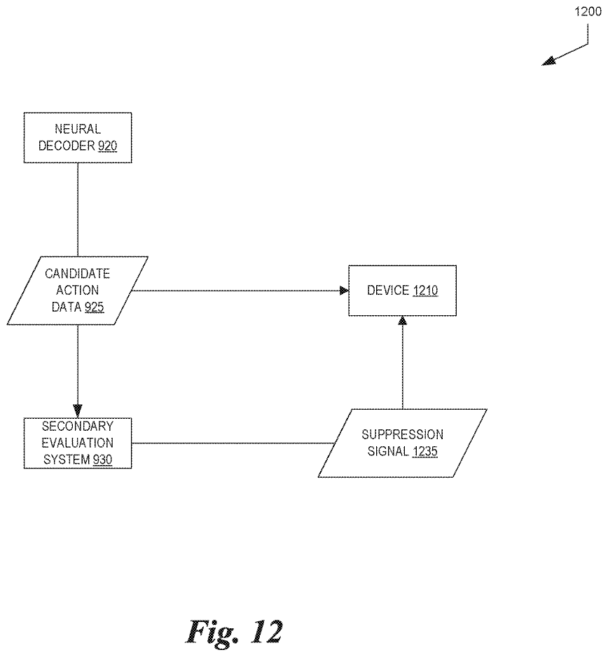

FIG. 12 shows a block diagram including example data flows 1200 for providing secondary evaluation of a candidate action. In this example, the neural decoder 920, secondary evaluation system 930, and device 1210 are in communication. The neural decoder 920 in this example provides candidate action data 925 to the device 1210 and secondary evaluation system 930. The secondary evaluation system 930 may transmit a suppression signal 1235 to the device 1210 to affect how the device 1210 responds to the candidate action data 925 received from the neural decoder 920.

The neural decoder 920, candidate action data 925, and secondary evaluation system 930, are as described in relation to FIG. 9 above herein.

The device 1210 may be any type of device, such as the user device 950 described above herein, or a robotic actuator 1410 as described below herein. The device 1210 is configured to receive candidate action data 925 from the neural decoder 920, or in some embodiments from the secondary evaluation system 930, and execute an action as indicated by the candidate action data 925. In some embodiments, the device 1210 may be configured to receive information in the format of a BCI HID protocol, and the candidate action data 925 and suppression signal 1235 may be formatted according to the BCI HID protocol used by the device 1210.

The suppression signal 1235 is a signal transmitted from the secondary evaluation system 930 to the device 1210 to cause the device 1210 to alter its response to the candidate action data 925, to stop the device 1210 from executing an action based on the candidate action data 925, or to cause the device 1210 to execute the action indicated in the candidate action data 925. To cause the device 1210 to not perform an action indicated by the candidate action data 925, the suppression signal 1235 may include an identifier for the candidate action data 925 (e.g., a unique identifier included as part of the candidate action data 925, a timestamp of the candidate action data 925, etc.), and provide to the device 1210 an instruction to ignore the identified candidate action data 925. The suppression signal 1235 may cause the device 1210 to alter an action indicated by the candidate action data 925 by referring to an identifier for the candidate action data 925 and including additional information for performing the action (e.g., the alteration to be applied to the action in the candidate action data 925) or may include replacement action data for the action data in the candidate action data 925. The suppression signal 1235 may identify candidate action data 925 using an identifier for the candidate action data 925 and include an instruction to execute the candidate action data 925 at a time indicated by the candidate action data 925 or at a different time. In some embodiments, such as where the secondary evaluation system 930 provides the candidate action data 925 to the device 1210, the suppression signal 1235 may be provided at a same time as the candidate action data 925, or may include the candidate action data 925 (e.g., in a combined data object transmitted to the device 1210).

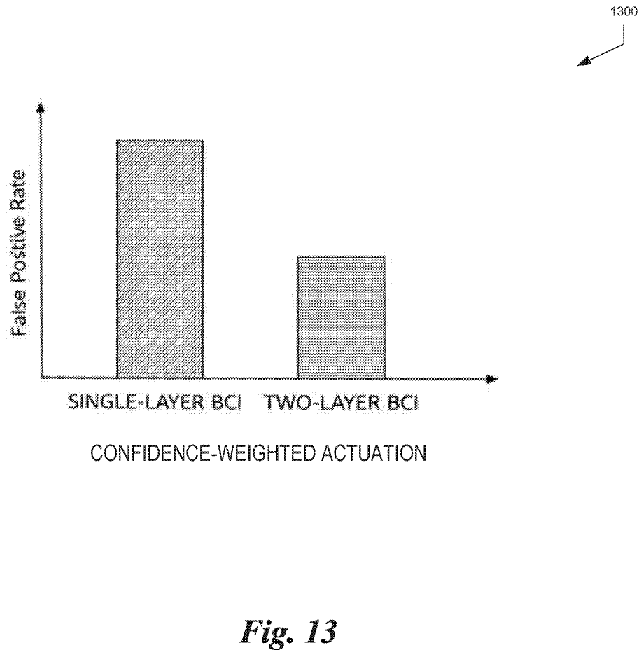

FIG. 13 includes a visualization of bar graph 1300 that shows a difference between a false positive rate of a single-layer BCI and a two-layer BCI. The relative false positive rate is represented as the y-axis of the bar graph 1300. As used herein, a single-layer BCI refers to a BCI that provides action information (e.g., the candidate action data 925 described above herein) to a user device 950 without an intermediate or additional evaluation by a system (e.g., the secondary evaluation system 930). As used herein, a two-layer BCI refers to a BCI that provides action information from a neural decoder 920 (e.g., as candidate action data 925) to a secondary evaluation system (e.g., the secondary evaluation system 930) for further analysis based on contextual data (e.g., the validation context data 927). As shown in this bar graph 1300, the false positive rate of a single-layer BCI may be substantially higher than that of a two-layer BCI due to the lack of additional analysis or additional information on which to base further analysis of the candidate action data 925.

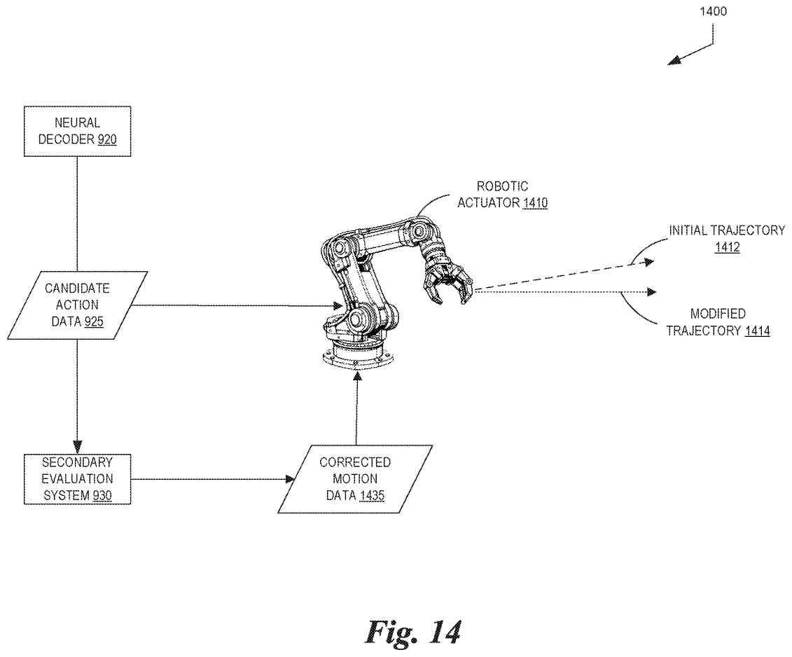

FIG. 14 shows an example environment 1400 for controlling a robotic actuator using a BCI system. The environment 1400 includes a neural decoder 920, secondary evaluation system 930, and robotic actuator 1410. In this example environment 1400, the robotic actuator 1410 is shown as being provided an initial trajectory 1412, and a modified trajectory 1414.

In this environment 1400, the neural decoder 920 generates candidate action data 925 provided to the robotic actuator 1410 and the secondary evaluation system 930. The candidate action data in this example environment 1400 is provided by the neural decoder 920 to the robotic actuator 1410, however in some embodiments, the candidate action data 925 may be provided to the robotic actuator 1410 via the secondary evaluation system 930. The candidate action data 925 of this example includes an initial trajectory 1412 along which the robotic actuator 1410 is to move. The initial trajectory 1412 may include movement of one or more motors of the robotic actuator 1410 to move in one or more directions (e.g., one-axis motion, two-axis motion, six-axis motion, etc.). The candidate action data 925 may include motor control signals, or may provide high-level (e.g., in a computing language) instructions to the robotic actuator 1410 that the robotic actuator 1410 interprets to cause motion of the robotic actuator 1410 along the initial trajectory 1412.

The secondary evaluation system 930 generates corrected motion data 1435 based on the candidate action data 925. The secondary evaluation system 930 may also use validation context data 927 to generate the corrected motion data 1435. The corrected motion data may indicate a new trajectory for the robotic actuator 1410 to place the robotic actuator 1410 in a location intended by the user, or to align movement of the robotic actuator 1410 along one or more intended axes of motion.

The candidate action data 925 and corrected motion data 1435 may include motor control signals for individual motors of the robotic actuator 1410, a direction of motion, magnitude of motion, length of time for which the robotic actuator 1410 is to move along one or more axes, a velocity profile, an intended end location in two-dimensional space (e.g., as defined within a plane of motion), an intended end location in three-dimensional space.

In some embodiments, the robotic actuator 1410 may begin motion along the initial trajectory 1412 before receiving corrected motion data 1435 to move along the modified trajectory 1414. In some embodiments, the secondary evaluation system 930 may generate the corrected motion data 1435 based on the robotic actuator 1410 having moved along the initial trajectory 1412 while the secondary evaluation system 930 generates the corrected motion data 1435. For example, the secondary evaluation system 930 may determine a time during which the robotic actuator 1410 has already moved along the initial trajectory 1412, and generate corrected motion data 1435 to move from the current position of the robotic actuator 1410 to a correct position intended by the user.

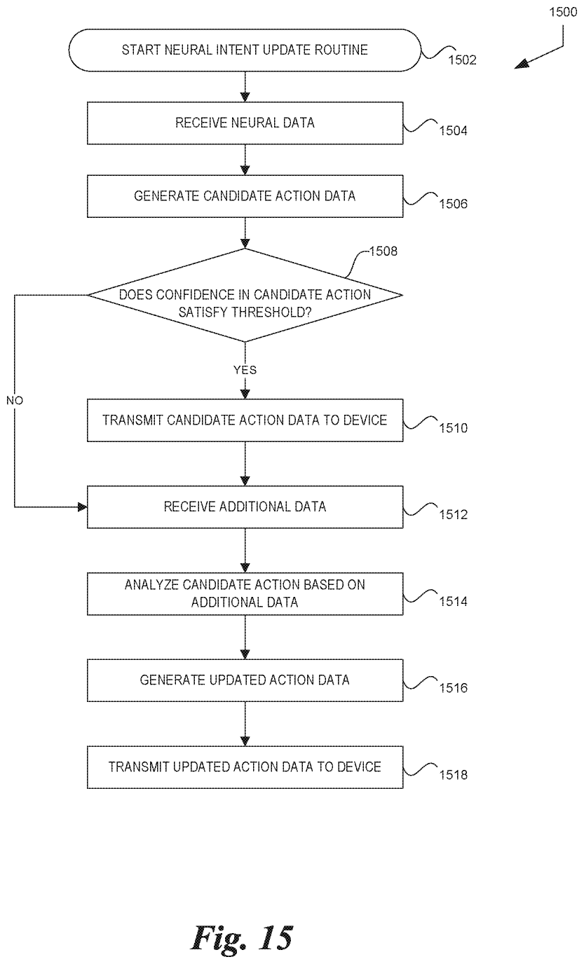

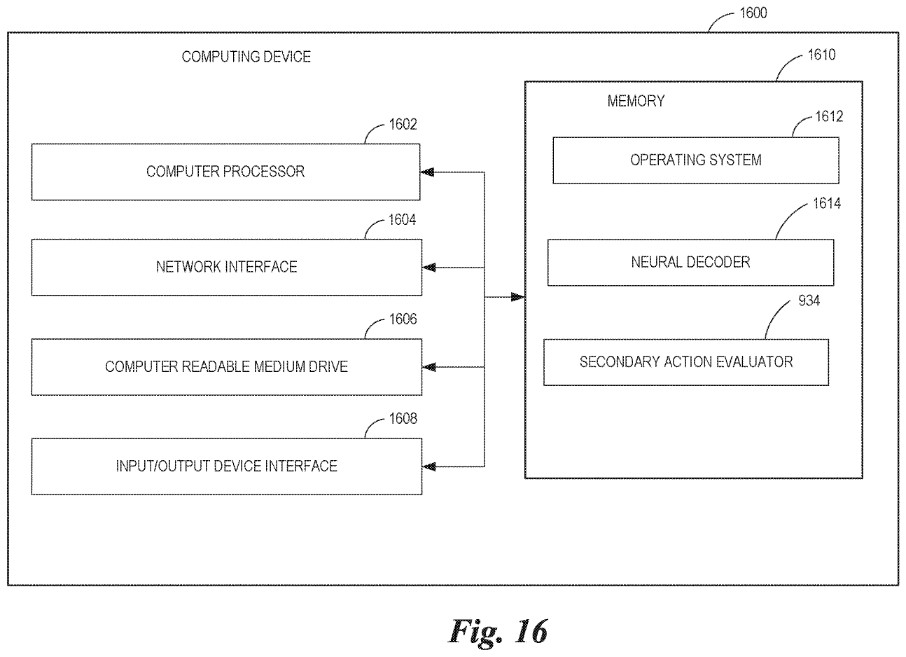

FIG. 15 illustrates example routine 1500 for updating a neural intent based on validation context data. When a routine described herein such as routine 1500 is initiated, a set of executable program instructions stored on one or more non-transitory computer-readable media (e.g., hard drive, flash memory, removable media, etc.) may be loaded into memory (e.g., random access memory or RAM) of a computing device, such as the computing device 1600 shown in FIG. 16, and executed (e.g., a computer-implemented method) by one or more processors. In some embodiments, the routine 1500, or portions thereof, may be implemented on multiple processors, serially or in parallel.

Further, routines described herein may refer, in part, to an order of operations, blocks, or steps. It should be understood that any reference to an order of the blocks of a routine is intended to aid in the explanation of the routine only, and does not imply that such an order is required for performing the operation described by the routine. Blocks of the routines described herein, therefore, should be understood to be capable of being performed before, after, and/or simultaneously with any other block of the routine. Additionally, various blocks of routines described herein may be performed repeatedly even where no loop is explicitly shown. Such loops may be excluded from the description of a routine for the purpose of clarity, and does not require that a block be performed only once before the routine proceeds.

The routine 1500 begins at block 1502, for example when a signal source 905 generates a neural signal recorded by the neural recording device 910.

At block 1504, the neural decoder 920 receives neural data from the neural recording device 910. The neural data may be based on signal information generated by the signal source 905 in response to a user forming an intent in their mind. The intent may be a motion intent (e.g., to move a body part). The generation of neural data by the neural decoder 920 is described above herein.

At block 1506, the neural decoder 920 generates candidate action data 925. The candidate action data 925 may be a description of an action to be performed by a user device 950. The candidate action data 925 may be formatted by the neural decoder 920 according to a BCI HID definition used by the user device 950. The candidate action data 925 may include motor control signals (e.g., as described in relation to FIG. 14 above herein). The candidate action data 925 may be formatted according to the user device 950 being controlled by the neural decoder 920. Optionally, the neural decoder 920 may include confidence data 923 in the candidate action data 925, or separately provide the confidence data 923. The confidence data 923 may represent a confidence output by one or more machine learning models implemented by the neural decoder 920 to generate the candidate action data 925 from the neural data received from the neural recording device 910. The candidate action data 925 and/or confidence data 923 may be transmitted by the neural decoder 920 directly to the secondary evaluation system 930 and user device 950. In some embodiments, the candidate action data 925 and/or confidence data 923 may be transmitted to the secondary evaluation system 930, and the secondary evaluation system 930 may provide the received data to the user device 950. In some embodiments, the candidate action data 925 may be transmitted to one or both of the secondary evaluation system 930 or user device 950 and the confidence data 923 may be transmitted to only one of the secondary evaluation system 930 or user device 950. For example, the neural decoder 920 may transmit the confidence data 923 only to the secondary evaluation system 930, and the secondary evaluation system 930 may determine for the user device 950 how to proceed based on the confidence data 923. In another example, the neural decoder 920 may transmit the confidence data 923 to the user device 950, and the user device 950 may or may not act based on whether the confidence data 923 satisfies a threshold.

In embodiments where the secondary evaluation system 930 receives confidence data and determines whether to transmit candidate action data 925 to the user device 950, at decision block 1508 the secondary evaluation system 930 optionally determines whether the confidence data 923 has satisfied a threshold value. Satisfying the threshold may be determined based on a numerical value of the confidence exceeding a threshold value, for example where the confidence indicates a likelihood the candidate action data 925 is accurate as determined by the neural decoder 920. Satisfying the threshold may be determined based on a numerical value of the confidence being lower than a threshold value, for example where the confidence indicates an uncertainty in the candidate action data 925 as determined by the neural decoder 920. The threshold may be fixed. The threshold may be dynamically determined. For example, the secondary evaluation system 930 may determine the threshold based on an action type indicated by the candidate action data 925. In another example, the secondary evaluation system 930 may determine the threshold based on a type of user device 950 to which the candidate action data 925 will be provided.

In some embodiments, the decision block 1508 may be performed by the neural decoder 920. For example, the determination of whether the candidate action data 925 satisfies the threshold may be used to determine whether the neural decoder 920 provides the candidate action data 925 directly to the user device 950, or transmits the candidate action data 925 to the secondary evaluation system 930 for further evaluation before an action is provided to the user device 950. Where the threshold is satisfied by the confidence data 923, the routine 1500 moves to block 1510. Where the threshold is not satisfied by the confidence data 923, the routine 1500 moves to block 1512.

At block 1510, in embodiments where the secondary evaluation system 930 provides the candidate action data 925 to the user device 950, in response to the confidence data 923 satisfying the threshold the secondary evaluation system 930 transmits the candidate action data 925 to the user device 950. In some embodiments, where the neural decoder 920 uses the threshold to determine whether to transmit the candidate action data 925 directly to the user device 950, the neural decoder 920 transmits the candidate action data 925 to the user device 950 and the secondary evaluation system 930.

At block 1512, the secondary evaluation system 930 receives validation context data 927. The validation context data 927 may be received from any data source, for example as described in relation to FIGS. 9 and 10 above herein. Different validation context data 927 may be received from different sources at different times. The secondary evaluation system 930 may associate the validation context data 927 from different sources based on a timestamp indicating when each validation context data 927 was generated. Different validation context data 927 may be associated if the validation context data 927 is generated at a same time, or within a same window of time (e.g., 50 ms, 750 ms, 2 s, etc.). The window of time may be a fixed window of time, or may be determined by the secondary evaluation system 930 (e.g., based on an action type of the candidate action data 925, type of user device 950 being controlled, etc.). The secondary data extractor 932, as described above herein, may parse the validation context data 927 to identify relevant information in the validation context data 927.

At block 1514, the secondary evaluation system 930 analyzes the candidate action based on the validation context data 927. The secondary action evaluator 934 may determine whether any of the validation context data 927 is relevant to the candidate action data 925. For example, if the candidate action data 925 indicates the user intends to select a button, and the user has a high heart rate, the secondary evaluation system 930 may determine whether the high heart rate is relevant to selecting the button. Additionally, the secondary action evaluator 934 may evaluate a first type of validation context data 927 in the context of a second type of validation context data 927. For example, a first validation context data 927 may indicate a user is in motion, and a second validation context data 927 may indicate the user's body is not moving. In this example, the secondary action evaluator 934 may determine the user is likely stationary within a moving vehicle and ignore the first validation context data 927 indicating the user is in motion.

At block 1516, the secondary action evaluator 934 generates updated action data based on the analysis of the candidate action data 925 and validation context data 927. The updated action data, as described above herein, may alter the candidate action data 925 or instruct the user device 950 to execute the candidate action data 925 as originally received.

At block 1518, The secondary evaluation system 930 transmits the updated action data to the user device 950. Where no alterations are made to the candidate action data 925, the secondary evaluation system 930 may transmit the original candidate action data 925 (e.g., in embodiments where the user device 950 receives candidate action data 925 from the secondary evaluation system 930 and not the neural decoder 920), may transmit an indication to proceed to execute the candidate action data 925, or may transmit nothing (e.g., where there is an latency window 1110 after which the user device 950 automatically executes a received candidate action data 925).

FIG. 16 illustrates various components of an example computing device 1600 configured to implement various functionality described herein.

In some embodiments, the computing device 1600 may be implemented using any of a variety of computing devices, such as server computing devices, desktop computing devices, personal computing devices, mobile computing devices, mainframe computing devices, midrange computing devices, host computing devices, or some combination thereof. In some embodiments, the computing device 1600 may be implemented partly or entirely as an implantable device that is implanted in a user's body.

In some embodiments, the features and services provided by the computing device 1600 may be implemented as web services consumable via one or more communication networks. In further embodiments, the computing device 1600 is provided by one or more virtual machines implemented in a hosted computing environment. The hosted computing environment may include one or more rapidly provisioned and released computing resources, such as computing devices, networking devices, and/or storage devices. A hosted computing environment may also be referred to as a “cloud” computing environment.

In some embodiments, as shown, a computing device 1600 may include: one or more computer processors 1602, such as physical central processing units (“CPUs”); one or more network interfaces 1604, such as a network interface cards (“NICs”); one or more computer readable medium drives 1606, such as a high density disk (“HDDs”), solid state drives (“SSDs”), flash drives, and/or other persistent non-transitory computer readable media; one or more input/output device interfaces 1608; and one or more computer-readable memories 1610, such as random access memory (“RAM”) and/or other volatile non-transitory computer readable media.

The computer-readable memory 1610 may include computer program instructions that one or more computer processors 1602 execute and/or data that the one or more computer processors 1602 use in order to implement one or more embodiments. For example, the computer-readable memory 1610 can store an operating system 1612 to provide general administration of the computing device 1600. As another example, the computer readable memory 1610 can store at least one neural decoder 1614 to decode neural signal information recorded by a recording device from a brain. As another example, the computer-readable memory 1610 can store a secondary action evaluator 934.

### Terminology

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or combinations of electronic hardware and computer software. To clearly illustrate this interchangeability, various illustrative components, blocks, modules, and steps have been described above generally in terms of their functionality. Whether such functionality is implemented as hardware, or as software that runs on hardware, depends upon the particular application and design conditions imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

As for other details of the present invention, materials and manufacturing techniques may be employed as within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described and equivalents (whether recited herein or not included for the sake of some brevity) may be substituted without departing from the true spirit and scope of the invention. Also, any optional feature of the inventive variations may be set forth and claimed independently or in combination with any one or more of the features described herein. Accordingly, the invention contemplates combinations of various aspects of the embodiments or combinations of the embodiments themselves, where possible. Reference to a singular item, includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

Examples of embodiments of the present disclosure can be described in view of the following clauses:

Clause 1. A method of assisting an individual using a brain-computer interface to engage an electronic device, the method comprising: transmitting an electronic signal from a neural interface device of the brain-computer interface to a processing component of the brain-computer interface, wherein the electronic signal is associated with neural activity of the individual from a first region in a brain of the individual and wherein the processing component is configured with a plurality of algorithms including a decoding algorithm and a supplemental algorithm; obtaining a secondary data comprising information associated with the individual and transmitting the secondary data to the processing component; decoding the electronic signal using the decoding algorithm to produce an output signal; and processing the secondary data using the supplemental algorithm to determine whether altering an effect of the output signal on the electronic device is required.

Clause 2. The method of clause 1, wherein altering the effect of the output signal comprises terminating transmission of the output signal to the electronic device.

Clause 3. The method of any of clauses 1-2, wherein altering the effect of the output signal comprises instructing the electronic device to ignore the output signal.

Clause 4. The method of any of clauses 1-3, wherein the first region of the brain comprises a motor cortex region of the brain.

Clause 5. The method of any of clauses 1-4, wherein the secondary data is associated with neural activity of the individual from a second region in the brain of the individual.

Clause 6. The method of clause 5, wherein the second region comprises a region selected from a group consisting of a supplementary motor cortex, a frontal cortex, a dorsal anterior cortex, and a pre-frontal cortex.

Clause 7. The method of clause 5, wherein the secondary data comprises a cognitive neural activity.

Clause 8. The method of any of clauses 1-7, wherein the secondary data comprises muscle movement data associated with the individual.

Clause 9. The method of any of clauses 1-8, wherein the supplemental algorithm includes a historical data recorded from the individual during use of the brain-computer interface.

Clause 10. The method of any of clauses 1-9, wherein the secondary data is contemporaneous with electronic signal.

Clause 11. The method of any of clauses 1-10, wherein obtaining the secondary data comprises obtaining the secondary data that coincides in time with the neural activity.

Clause 12. The method of any of clauses 1-11, wherein the secondary data is detected using the neural interface device.

Clause 13. A method of assisting an individual using a brain-computer interface to engage an electronic device, the method comprising: transmitting an electronic signal from a neural interface device of the brain-computer interface to a processing component of the brain-computer interface, wherein the electronic signal is associated with neural activity of the individual from a first region in a brain of the individual and wherein the processing component is configured with a plurality of algorithms including a decoding algorithm and a supplemental algorithm; obtaining a secondary data comprising information associated with the individual and transmitting the secondary data to the processing component; decoding the electronic signal using the decoding algorithm to produce an output signal; and applying the supplemental algorithm to the secondary data to determine whether to alter the output signal prior to transmitting the output signal to an electronic device coupled to the brain-computer interface.

Clause 14. The method of clause 13, wherein altering the effect of the output signal comprises terminating transmission of the output signal to the electronic device.

Clause 15. The method of any of clauses 13-14, wherein altering the effect of the output signal comprises instructing the electronic device to ignore the output signal.

Clause 16. The method of any of clauses 13-15, wherein the first region of the brain comprises a motor cortex region of the brain.

Clause 17. The method of any of clauses 13-16, wherein the secondary data is associated with neural activity of the individual from a second region in the brain of the individual.

Clause 18. The method of clause 17, wherein the second region comprises a region selected from a group consisting of a supplementary motor cortex, a frontal cortex, a dorsal anterior cortex, and a pre-frontal cortex.

Clause 19. The method of clause 17, wherein the secondary data comprises a cognitive neural activity.

Clause 20. The method of any of clauses 13-19, wherein the secondary data comprises muscle movement data associated with the individual.

Clause 21. The method of any of clauses 13-20, wherein the supplemental algorithm includes a historical data recorded from the individual during use of the brain-computer interface.

Clause 22. The method of any of clauses 13-21, wherein the secondary data is contemporaneous with electronic signal.

Clause 23. The method of any of clauses 13-22, wherein obtaining the secondary data comprises obtaining the secondary data that coincides in time with the neural activity.

Clause 24. The method of any of clauses 13-23, wherein the secondary data is detected using the neural interface device.

Clause 25. A computer-implemented method for secondary evaluation of candidate action data, the computer-implemented method comprising: receiving candidate action data indicating an intended action of a user derived from decoded neural signal data of the user; receiving validation context data comprising at least one of: environmental data associated with an environment in which the user is located, location data associated with a location or movement of the user, behavioral data associated with prior behaviors of the user, or physiological data representing a physical condition of the user; evaluating the candidate action data based on the validation context data to determine whether to alter the candidate action data; generating modified action data based on the candidate action data and the evaluation; and transmitting the modified action data to a computing device to cause the computing device to execute an action.

Clause 26. The computer-implemented method of clause 25, wherein the validation context data is generated within a time window associated with a time at which the candidate action data is received.

Clause 27. The computer-implemented method of any of clauses 25-26, wherein the validation context data is generated within a time window associated with a time at which the candidate action data is generated.

Clause 28. The computer-implemented method of any of clauses 25-27, wherein generating modified action data comprises altering a type of action of the candidate action data.

Clause 29. The computer-implemented method of any of clauses 25-28, wherein generating modified action data comprises modifying a length of time during which a candidate action of the candidate action data is to be performed by the computing device.

Clause 30. The computer-implemented method of any of clauses 25-29, further comprising transmitting the candidate action data to the computing device.

Clause 31. The computer-implemented method of clause 30, wherein the validation context data is received before transmitting the candidate action data to the computing device.

Clause 32. The computer-implemented method of clause 30, wherein the validation context data is received after transmitting the candidate action data to the computing device.

Clause 33. The computer-implemented method of clause 30, wherein the modified action data is a suppression signal, and wherein transmitting the modified action data to the computing device causes the computing device to stop executing the action.

Clause 34. The computer-implemented method of clause 30, wherein the candidate action data comprises a confidence value, and wherein the computer-implemented method further comprises: determining the confidence value satisfies a threshold confidence value, and wherein transmitting the candidate action data to the computing device is based on the confidence value satisfying the threshold confidence value.

Clause 35. The computer-implemented method of any of clauses 25-34, wherein evaluating the candidate action data based on the validation context data to determine whether to alter the candidate action data comprises: applying the candidate action data and the validation context data as input to a machine learning model to cause the machine learning model to generate a determination as to whether a candidate action of the candidate action data is accurate.

Clause 36. The computer-implemented method of clause 35, wherein the modified action data is generated in response to the machine learning model determining the candidate action is not accurate.

Clause 37. The computer-implemented method of clause 35, wherein the machine learning model further generates an indication of a corrected action.

Clause 38. The computer-implemented method of clause 37, wherein generating the modified action data is further based on the indication of the corrected action.

Clause 39. The computer-implemented method of any of clauses 25-38, wherein the modified action data causes the computing device to delay execution of a candidate action associated with the candidate action data.

Clause 40. A system comprising: a neural decoder comprising a computer processor, the neural decoder configured to generate candidate action data from a neural intent signal of a user, wherein the candidate action data represents an action to be executed by a computing device; a secondary data extractor comprising a computer processor, the secondary data extractor configured to obtain validation context data independent of the neural intent signal; and a secondary action evaluator comprising a computer processor, the secondary action evaluator configured to generate an alteration to the candidate action data based on the validation context data, the alteration configured to alter an effect of the candidate action data on the computing device.

Clause 41. The system of clause 40, wherein the validation context data comprises at least one of: environmental data associated with an environment in which the user is located, location data associated with a location or movement of the user, behavioral data associated with prior behaviors of the user, or physiological data representing a physical condition of the user.

Clause 42. The system of any of clauses 40-41, wherein the validation context data is generated within a time window associated with a time at which the candidate action data is received.

Clause 43. The system of any of clauses 40-42, wherein the neural decoder is further configured to transmit the candidate action data to the computing device.

Clause 44. The system of any of clauses 40-42, wherein the secondary action evaluator is further configured to transmit the candidate action data to the computing device.

What is claimed is:

1. A computer-implemented method for secondary evaluation of candidate action data, the computer-implemented method comprising:
receiving candidate action data indicating an intended action of a user derived from decoded neural signal data of the user;
receiving validation context data comprising at least one of: environmental data associated with an environment in which the user is located, location data associated with a location or movement of the user, behavioral data associated with prior behaviors of the user, or physiological data representing a physical condition of the user;
evaluating the candidate action data based on the validation context data to determine whether to alter the candidate action data;
generating modified action data based on the candidate action data and the evaluation; and
transmitting the modified action data to a computing device to cause the computing device to execute an action.

2. The computer-implemented method of claim 1, wherein the validation context data is generated within a time window associated with a time at which the candidate action data is received.

3. The computer-implemented method of claim 1, wherein the validation context data is generated within a time window associated with a time at which the candidate action data is generated.

4. The computer-implemented method of claim 1, wherein generating modified action data comprises altering a type of action of the candidate action data.

5. The computer-implemented method of claim 1, wherein generating modified action data comprises modifying a length of time during which a candidate action of the candidate action data is to be performed by the computing device.

6. The computer-implemented method of claim 1, further comprising transmitting the candidate action data to the computing device.

7. The computer-implemented method of claim 6, wherein the validation context data is received before transmitting the candidate action data to the computing device.

8. The computer-implemented method of claim 6, wherein the validation context data is received after transmitting the candidate action data to the computing device.

9. The computer-implemented method of claim 6, wherein the modified action data is a suppression signal, and wherein transmitting the modified action data to the computing device causes the computing device to stop executing the action.

10. The computer-implemented method of claim 6, wherein the candidate action data comprises a confidence value, and wherein the computer-implemented method further comprises:
determining the confidence value satisfies a threshold confidence value, and
wherein transmitting the candidate action data to the computing device is based on the confidence value satisfying the threshold confidence value.

11. The computer-implemented method of claim 1, wherein evaluating the candidate action data based on the validation context data to determine whether to alter the candidate action data comprises:
applying the candidate action data and the validation context data as input to a machine learning model to cause the machine learning model to generate a determination as to whether a candidate action of the candidate action data is accurate.

12. The computer-implemented method of claim 11, wherein the modified action data is generated in response to the machine learning model determining the candidate action is not accurate.

13. The computer-implemented method of claim 11, wherein the machine learning model further generates an indication of a corrected action.

14. The computer-implemented method of claim 13, wherein generating the modified action data is further based on the indication of the corrected action.

15. The computer-implemented method of claim 1, wherein the modified action data causes the computing device to delay execution of a candidate action associated with the candidate action data.

16. A system comprising:
a neural decoder comprising a computer processor, the neural decoder configured to generate candidate action data from a neural intent signal of a user, wherein the candidate action data represents an action to be executed by a computing device;
a secondary data extractor comprising a computer processor, the secondary data extractor configured to obtain validation context data independent of the neural intent signal; and
a secondary action evaluator comprising a computer processor, the secondary action evaluator configured to generate an alteration to the candidate action data based on the validation context data, the alteration configured to alter an effect of the candidate action data on the computing device.

17. The system of claim 16, wherein the validation context data comprises at least one of: environmental data associated with an environment in which the user is located, location data associated with a location or movement of the user, behavioral data associated with prior behaviors of the user, or physiological data representing a physical condition of the user.

18. The system of claim 16, wherein the validation context data is generated within a time window associated with a time at which the candidate action data is received.

19. The system of claim 16, wherein the neural decoder is further configured to transmit the candidate action data to the computing device.

20. The system of claim 16, wherein the secondary action evaluator is further configured to transmit the candidate action data to the computing device.
